(19) United States
(12) Patent Application Publication
(10) Pub. No.: US 2014/0213465 A1
(43) Pub. Date: Jul. 31, 2014

(54) PLAQUE ARRAY METHODS AND COMPOSITIONS FOR FORMING AND DETECTING PLAQUES

(71) Applicant: PLAXGEN INC, Sunnyvale, CA (US)

(72) Inventor: Shanmugavel Madasamy, Cupertino, CA (US)

(73) Assignee: PLAXGEN, INC., SUNNYVALE, CA (US)

(21) Appl. No.: 13/684,027

(22) Filed: Nov. 21, 2012

Related U.S. Application Data
(60) Provisional application No. 61/566,580, filed on Dec. 2, 2011.

(51) Int. Cl. G01N 33/92; G01N 33/53

(52) U.S. Cl. CPC G01N 33/92; G01N 33/53
USPC 506/9

(57) ABSTRACT

Provided herein are methods and compositions for the in vitro formation of an array of plaque particles for use in biological assays, diagnosis, drug discovery and drug development. More specifically, the embodiments described herein relate to the in vitro synthesis of plaque particles when treated with biofluids and identification of such plaque particles by a variety of detection systems. In particular, the resulting in vitro plaque particles resemble atherosclerotic and amyloid plaques. The plaque embodiments described may be used to enable rapid, sensitive and/or efficient drug discovery, medical diagnostics and patient stratification.

### FIELD OF THE INVENTION

The methods and compositions described herein enable rapid, sensitive and/or efficient in vitro diagnosis, biotechnology tools and drug discovery and development of plaque-associated diseases.

### BACKGROUND

Atherosclerosis is a complex, progressive and chronic inflammatory cardiovascular disease caused by assembly and progression of atherosclerotic plaque in the arteries (Lippy P et al 2011). According to American Heart Association, approximately, 40 million people in US are believed to have atherosclerosis without noticeable clinical symptoms and only 6 million are symptomatic. A number of analytical methods and tools are used to diagnose both symptomatic and asymptomatic subjects of the atherosclerosis (Naghavi et al, 2003)

TABLE 1
Analytical tools/methods commonly used
for diagnosis of atherosclerosis

| | Diagnostic | |
|---|---|---|
| # | method/tools | Specific aim |
| 1 | Cardiac | to locate the narrowing, occlusions, and |
| | catheterization | other abnormalities of specific arteries |
| 2 | Computed tomography | to diagnose and analyze the presence of |
| | | calcified nodules in the atherosclerotic |
| | | plaques |
| 3 | X-ray diffraction | to identify and analyze the presence of |
| | | crystalline contents of cholesterol and |
| | | calcium phosphate |
| 4 | Optical microscopy | for semi-quantitative analysis of |
| | and/or Raman | crystalline contents of cholesterol |
| | spectroscopy | and calcium in the plaques |
| 5 | Doppler sonography | a special transducer is used to direct |
| | | sound waves into a blood vessel to |
| | | evaluate blood flow |
| 6 | MUGA/radionuclide | Nuclear scan to see how the heart wall |
| | angiography | moves and how much blood is expelled |
| | | with each heartbeat |
| 7 | Homocysteine | an amino acid marker in the blood that, |
| | | at high levels, may damage the lining |
| | | of the arterial wall |
| 8 | Lipoprotein (a) | a unique lipid, or fat, often elevated |
| | | in people who have a family history of |
| | | early-onset atherosclerosis |
| 9 | Small LDL particles | a predominance of small particles of |
| | | LDL, or “bad,” cholesterol that may |
| | | form plaque in the arteries, causing |
| | | atherosclerosis more easily than larger |
| | | LDL particles |
| 10 | C-Reactive protein | a trace protein that is a marker for |
| | | inflammation and is associated with |
| | | higher risk of heart attack and stroke |
| 11 | Electrocardiogram | is a test that measures the electrical |
| | | activity of the heartbeat |

Most of these diagnostic procedures and techniques are expensive and invasive often involving administration of chemical and/or radioactive compounds to localize or visualize the atherosclerotic plaques (Greenland et al, 2007). In addition, the results obtained from these procedures are not sufficient enough to conclusively suggest therapeutic intervention to the suspected patients. Further, limited knowledge about the mechanism of atherosclerotic plaque formation make drug discovery and development challenging. Thus, there is a need for simple, determinative, cost-effective solutions for the diagnosis, drug discovery and development of plaque-associated diseases such as atherosclerosis.

Amyloidosis are a group of more than twenty plaque-associated diseases characterized by protein aggregation including Alzheimer's Disease (AD), Parkinson's Disease (PD), prion-mediated diseases, Huntington disease (HD), Multiple sclerosis (MS), type 2 diabetes and the like. AD is a common neurodegenerative disease associated with progressive dementia caused mostly due to the deposition of Amyloid-beta (Abeta) peptides (Yankner, 1996). Abnormal processing of the Abeta precursor protein is an early and causative event in the pathogenesis of AD (Selkoe D J, 2003). Abeta peptides released from amyloid precursor protein by the action of β- and γ-secretases undergo structural transformation from monomers to oligomers and finally into amyloid fibrils/plaques (Dobson C M, 2003).

The pathological consequences of such senile plaque accumulation are neuronal loss, cerebrovascular inflammation, reduction in the cerebrovascular space and cognitive decline (Bell R D et al, 2009).

AD is definitively diagnosed through examination of brain tissue, usually at autopsy (Khachaturian et al 1985; McKhann et al, 1984). Post-mortem slices of brain tissue of subjects with AD exhibit the presence of amyloid in the form of proteinaceous extracellular cores of the neuritic plaques that are characteristic of AD.

Research efforts to develop methods for diagnosing AD include (1) genetic testing, (2) immunoassay methods and (3) imaging techniques. The limitations of these methods are several-fold. Genetic analysis of a large number of AD families has demonstrated that AD is genetically heterogeneous (George-Hyslop et al, 1990). Also, the genetic tests reveal risk factors rather than disease markers for AD. The immunoassay methods diagnosing presence of amyloid related protein in cerebrospinal fluid (CSF) for diagnosing AD have not been proven to detect AD in all patients, particularly at early stages of the disease. Imaging methods face the challenge of getting the imaging agent such as antibodies or radio-labeled peptides across the blood brain barrier. Identifying asymptomatic AD individuals is a challenging task and multiple tests involving neurophysiological and neuropathological techniques are currently used to diagnose these subjects (Thies W et al 2012). Clinical diagnosis of AD is not always accurate since the criteria are relatively subjective and the disease needs to be differentiated from other dementing illnesses. According to new recommendation from the National Institute on Aging and the Alzheimer's disease association, identification of novel biomarker is imperative both for AD diagnosis and drug discovery (McKhann G M et al, 2011; Sperling R A et al, 2011). The diagnosis of asymptomatic subjects in will help to initiate early therapeutic intervention to reduce life threatening risk associated with the progression of plaque-associated diseases including atherosclerosis and amyloidoses.

Accordingly, there remains an unmet need for novel, cost effective AD diagnostic methods and effective drug discovery and development technologies.

### INCORPORATION BY REFERENCE

All publications, patents, and patent applications mentioned in this specification are herein incorporated by reference to the same extent as if each individual publication, patent, or patent application was specifically and individually indicated to be incorporated by reference.

### SUMMARY OF THE INVENTION

In some embodiments, the present invention provides a method of detecting plaque particle formation in a subject, the method comprising: preparing at least one plaque aggregate or self-formed plaque particle in vitro wherein the plaque aggregate or self-formed plaque particle is linked to a detectable label; contacting a biological sample from the subject with the at least one plaque aggregate or self-formed plaque particle; and then employing a device to detect the detectable label. In some embodiments, the label is a fluorescent label or luminescent label or dye. In some embodiments, the device is a flow cytometer or other fluorescence detector or luminescent detector or colorimeter.

In some embodiments, the biological sample is a biological fluid. In other embodiments, the biological fluid is selected from the group consisting of: blood, plasma, serum, cerebral spinal fluid, urine and saliva. In some embodiments, the contacting of biological sample results in addition of components to the at least one plaque aggregate or self-formed plaque particle such that at least one plaque particle is formed. In some embodiments, the contacting results in addition of components to the at least one plaque aggregate or self-formed plaque particle such that at least one plaque particle is formed and the plaque particles formed resembles a plaque associated with atherosclerosis, Alzheimer's disease, Autism, Parkinson's disease, multiple sclerosis, osteoarthritis, Mad Cow Sponsiform, Type II diabetes, dementia, systemic amyloidosis, dialysis-related amyloidosis, lysozyme amyloidosis, insulin-related amyloidosis, and/or amyotrophic lateral sclerosis.

In some embodiments, the at least one plaque particle formed is compared to a plurality of self-formed plaque particles. In other embodiments, the subject is identified as having, or being at risk of having, a plaque-associated disease if the at least one plaque particle is substantially similar to a self-formed plaque particle among the plurality of self-formed plaque particles. In some embodiments, the plaque-associated disease is atherosclerosis or amyloidosis. In some embodiments the subject has, is at risk of having, or is suspected of having, atherosclerosis or an amyloidosis including Alzheimer's disease. In some embodiments, the at least one plaque aggregate or a plurality of plaque aggregates are used. In some embodiments the method further comprises diagnosing or stratifying subjects based on plaque particle formation, plaque particle sub-types, plaque particle images, plaque particle count, or plaque particle profile.

In some embodiments, at least one plaque aggregate or self-formed plaque particle comprises one or more of the following: protein, protein derivative, cholesterol, cholesterol derivative, lipid, lipid derivative, Abeta-42, Abeta derivatives, Synuclein, prion, Amylin, Tau, phospholipids, cholesterol crystals, Serum Amyloid A, Beta Microglobulin, lysozyme, insulin, or super dioxide dismutase, and calcium-phosphate (CP).

In some embodiments, the method further comprises screening the biological sample against a plurality of plaque aggregates or a pair of plaque aggregates labeled with different fluorophores for generating fluorescence resonance energy transfer (FRET) or a plurality of self-formed plaque particles or a pair of self-formed plaque particles labeled with different fluorophores for generating fluorescence resonance energy transfer (FRET).

In some embodiments, the method further comprises monitoring the subject by repeating steps of preparing plaque aggregates or self-formed plaque particles linked to a detectable label, contacting biological sample to plaque aggregates or self-formed plaque particles and using a device to detect a detectable label at different points over time. In some embodiments, the invention provides a method for detecting plaque particle formation in a subject, comprises: preparing at least one plaque aggregate or self-formed plaque particle; contacting a biological sample from the subject with the at least one plaque aggregate or self-formed plaque particle; then contacting the product with detectable label or an antibody-linked detectable label; and then employing a device to detect the detectable label.

In some embodiments, the invention provides a method of screening a test agent comprise: preparing the at least one plaque aggregate or self-formed plaque particle in vitro wherein the plaque aggregate or self-formed plaque particle is linked to a detectable label; contacting the at least one plaque aggregates or self-formed plaque particle linked to a detectable label with at least one test agents; and then employing a device to detect the detectable label. In some embodiments, the at least one of test agent comprises a small molecule or protein or antibody library of test agents. In some embodiments, the effect of at least one test agent is to accelerate the formation of plaque particles. In other embodiments, the effect of at least one test agent is to reduce or slow or disrupt plaque particle formation. In yet other embodiments, the method further comprises identifying the test agents that prevent or disrupt or reduce plaque particle formation. In other embodiments, the method further comprises testing the efficacy of the test agent or agents at disrupting plaque particles or reducing the formation of plaque particles or further comprising testing the safety of the test agent. In some embodiments, the test agent is a nanoparticle or is formulated with a nanoparticle. In these embodiments, the method further comprises monitoring the efficacy of the test agent in subjects.

In some embodiments, the invention provides a method of screening a test agent comprising: preparing at least one plaque aggregate or self-formed plaque particle in vitro wherein the at least one plaque aggregate or self-formed plaque particle is linked to a detectable label; culturing mammalian cells with the at least one plaque aggregate or self-formed plaque particle linked to a detectable label wherein the mammalian cells express morphologic changes, pathological symptoms, cell adhesion molecules, cytokines and or apoptosis, inflammation; contacting the mammalian cells at least one test agent; and then identifying test agents that prevent or lessen the formation of pathological symptoms or morphological changes in the cells.

In some embodiments, the invention provides a method of biomarker identification in a subject, the method comprising: preparing at least one plaque aggregate or self-formed plaque particle in vitro wherein the plaque aggregate or self-formed plaque particle is linked to a detectable label; contacting a biological sample from the subject with the at least one plaque aggregate or self-formed plaque particle; and then identification from of a protein or antibody or metabolite or substance in the biological sample that contributed to accelerated plaque particle formation using proteomics or mass spectrometry analysis or the like.

In some embodiments, the invention provides the methods disclosed above are used in the screening blood or blood products for plaque particle formation. In other embodiments, the blood or blood products is administered to a recipient subject following the screening or testing wherein the negative result is a finding of few or no new plaques following the contacting of said blood or blood product with the plaque aggregates.

### BRIEF DESCRIPTION OF THE DRAWINGS

A better understanding of the features and advantages of the present invention will be obtained by reference to the following detailed description that sets forth illustrative embodiments, in which the principles of the invention are utilized, and the accompanying drawings of which:

FIG. 1 represents a schematic of the plaque array method using a flow cytometer detection (Example 1).

FIG. 2A represents the three major steps involved in the in vivo plaque development process. First, the components of the plaque occur as molecules. Second, the components come together to form plaque aggregates. Third, the plaque aggregates transform into insoluble plaque particles. FIG. 2B illustrates the chemical structure of cholesterol derivatives. FIG. 2C illustrates the chemical structure of phospholipid derivatives.

FIG. 3 shows fluorescently-labeled cholesterol plaque aggregates and self-formed plaque particles are detectable by flow cytometry (Example 2).

In FIG. 4A, the plaque array method with flow cytometric detection showing serum of subjects with atherosclerosis accelerates the synthesis of plaque particles from cholesterol plaque aggregates (Example 3). FIG. 4B shows a time course study of cholesterol plaque particle formation from fluorescently-labeled plaque aggregates treated with normal and atherosclerotic subject serum samples. It is a plot of the number of cholesterol plaque particles (y-axis) versus time (x-axis): fluorescently-labeled cholesterol plaque aggregates incubated with serum from normal subjects (diamond); fluorescently-labeled cholesterol plaque aggregates incubated with serum from subjects with atherosclerosis (triangle, cross and squares). (Example 3).

In FIG. 5, the plaque array method with flow cytometric detection shows serum of subjects with atherosclerosis accelerates the synthesis of plaque particles from a small number of self-formed plaque particles. Plot A displays results from self-formed cholesterol particles whereas plots B-H display the results from self-formed plaque particles incubated for 1 hr with serum from seven subjects with atherosclerosis (Example 4).

In FIG. 6 the plaque array method with flow cytometric detection shows serum of subjects with atherosclerosis accelerates the synthesis of plaque particles from phospholipid plaque aggregates. Plot A displays fluorescently-labeled phospholipid plaque aggregates incubated for 1 hr. Plot B displays fluorescently-labeled phospholipid plaque aggregates incubated for 1 hr with serum from normal subjects. Plots C-F display results of fluorescently-labeled phospholipid plaque aggregates incubated for 1 hr with serum from four different subjects with atherosclerosis. (Example 5).

In FIG. 7, the plaque array method with flow cytometric detection shows results from incubation of cholesterol plaque aggregates with IgG-depleted serum of subjects with atherosclerosis results in less plaque particle formation compared with untreated serum. Flow cytometric analysis of fluorescently-labeled cholesterol plaque aggregates incubated for 1 hr with: (left) untreated serum from subject with atherosclerosis; (middle) IgG-depleted serum i.e. serum from subject with atherosclerosis that was pretreated with protein A/G and (right) IgG-depleted serum i.e. serum from subject with atherosclerosis that was pretreated with protein A (Example 6).

In FIG. 8, the plaque array method with flow cytometric detection used to detect in vivo changes in atherosclerotic mouse model. Mice carrying ApoE-gene mutation and normal C57BL/6 mice of the same age group were fed with atherogenic diet from 8 weeks to 20 weeks. The figures show results from flow cytometric detection of fluorescently-labeled cholesterol plaque aggregates after 1 hr incubation with serum samples collected at: (left to right) 8 weeks, 12 weeks, 16 weeks and 20 weeks from ApoE-mutant mice (top row) and C57BL/6-mice (bottom row) (Example 7).

In FIG. 9, the plaque array method with flow cytometric detection shows plaque particles synthesized from fluorescently-labeled Abeta-42 plaque aggregates treated with serum of subjects with AD. Plot A displays fluorescently-labeled Abeta-42 plaque aggregates incubated for 1 hr. Plot B displays fluorescently-labeled Abeta-42 plaque aggregates incubated for 1 hr with serum from normal subjects. Plots C-H display results of fluorescently-labeled Abeta-42 plaque aggregates incubated for 1 hr with serum from six different subjects with AD (Example 8).

In FIG. 10A, the plaque array method with flow cytometric detection shows serum of subjects with AD accelerates the formation of plaque particles from unlabeled Abeta-42 and Abeta-28 plaque aggregates (Example 9). In FIG. 10B, the plaque array method with flow cytometric detection shows serum of subjects with AD accelerates the formation of plaque particles from fluorescently-labeled Abeta-42 (left), Abeta-28 (middle) and cholesterol (right) plaque aggregates (Example 9).

In FIG. 11, the plaque array method with flow cytometric detection shows incubation of Abeta-42 plaque aggregates with IgG-depleted serum of subjects with AD shows reduced plaque particle formation compared with untreated serum. Flow cytometric analysis of unlabeled Abeta-42 plaque aggregates incubated for 1 hr with: (left) serum from subject with AD; (middle) IgG-depleted serum i.e. serum from subject with AD that was pretreated with protein A/G and (right) IgG-depleted serum i.e. serum from subject with AD that was pretreated with protein A. The plaque particles were detected using Thioflavin S dye (Example 10).

In FIG. 12, the plaque array method with flow cytometric detection used to detect in vivo changes in Alzheimer's mouse model. The results from flow cytometric detection of fluorescently-labeled cholesterol plaque aggregates after 1 hr incubation with serum samples collected at (left to right) 8 weeks, 12 weeks, 16 weeks and 20 weeks from APPSWE/PS-1 mutant mice (top row) and C57BL/6-mice (bottom row) of the same age group fed on a normal diet from 8 weeks to 20 weeks (Example 11).

FIG. 13A shows a bar graph depicting the results in relative fluorescence units (RFl) of an end point FRET assay of the effect of serum from subjects with AD on the synthesis of plaque particles from fluorescent-labeled Abeta-42 plaque aggregates (Example 12). FIG. 13B shows a bar graph depicting the results in relative fluorescence units (RFl) of an end point FRET assay of the effect of serum from subjects with atherosclerosis on the synthesis of plaque particles from cholesterol plaque aggregates (Example 13).

FIG. 14A represents the results from imaging flow cytometry which shows images and size distribution of Abeta-42 plaque particles synthesized from Abeta-42 plaque aggregates in the presence of serum from subjects with AD. The results indicate the existence of three major species of Abeta-42 plaque particles (Example 14). FIG. 14B represents the results from imaging flow cytometry which shows images and size distribution of cholesterol plaque particles synthesized from cholesterol plaque aggregates in the presence of serum from subjects with atherosclerosis. The results indicate the existence of three major species of cholesterol plaque particles (Example 15).

FIG. 15A shows three sub-types of cholesterol plaque particles synthesized from fluorescently-labeled cholesterol plaque aggregates under various conditions (Example 16). FIG. 15B shows two sub-types of phospholipid plaque particles synthesized from fluorescently-labeled phospholipid plaque aggregates under various conditions (Example 16).

FIG. 16 shows dot plots of phage display libraries panned with fluorescently-labeled Abeta-42 plaque aggregates (Example 17).

FIG. 17 shows flow cytometric analysis of Human Coronary Artery Endothelial Cells (HCAECs) treated with fluorescently-labeled cholesterol and phospholipid plaque aggregates (Example 18). The left plot shows forward and side scattering of cells treated with the fluorescent plaque aggregates. The middle plot shows the cells fluorescent detected in FL1 (520 nm) and FL2 (560 nm) channels of flow cytometer. The right histogram shows fluorescence intensity of cells bound with plaque aggregates.

FIG. 18 shows an apoptosis assay based on flow cytometric analysis of Human Coronary Artery Endothelial Cells (HCAECs) treated with fluorescently-labeled plaque aggregates. A. is a dot plot of HCAECs B. is a dot plot of HCAECs incubated with hybrid fluorescently-labeled cholesterol phospholipid plaque aggregates Ch1-LS.C. is a dot plot of HCAECs incubated with hybrid fluorescently-labeled calcium phosphate cholesterol phospholipid plaque hybrid aggregates CP-Ch1-LS D. shows a histogram of Annexin V binding to CP-Ch1-LS hybrid plaque treated HCAECs and E. shows a histogram of binding of propidium iodide to the CP-Ch1-LS hybrid plaque treated HCAECs (Example 19).

### DETAILED DESCRIPTION OF EMBODIMENTS OF THE INVENTION

As used herein, the singular forms “a,” “an” and “the” include plural referents unless the context clearly dictates otherwise. Abeta-42 refers to Amyloid beta peptide 1-42 and derivatives; Abeta-28 refers to Amyloid beta peptide 1-28 and derivatives; Abeta-17 refers to Amyloid beta peptide 1-17 and derivatives; Ch1 refers to cholesterol; LS refers to phospholipid; CP refers to calcium phosphate.

Two reagents used in the plaque array method are plaque aggregates and self-formed plaque particles. Plaque aggregates are prepared from organic or inorganic molecules under conditions that cause the molecules to aggregate. Plaque aggregates may be used directly in the plaque array method. For example, they could be used to screen a biological sample to diagnose if the subject has a plaque-associated disease. The second reagent disclosed herein is self-formed plaque particles. Self-formed plaque particles are formed from plaque aggregates. This occurs over time without contacting the plaque aggregates with biological sample. Like the plaque aggregates, the self-formed plaque particles can be used to screen a biological sample in the plaque array method. To perform the plaque array method biological sample is added to the reagent—either plaque aggregates or self-formed plaque particles. The addition of biological sample to the reagent results in formation of plaque particles which are detected by fluorescence or luminescence or colorimetry. The resulting plaque particles are referred to as plaque particles or in vitro plaque particles.

The “plaque particles” and “in vitro plaque particles” disclosed herein refer to the same reaction product formed in the presence of added biological sample and the terms are used interchangeably. These terms are different from the term “self-formed plaque particles” which are formed in the absence of added biological sample. “Self-formed plaque particles” refers to one type of reagent used in the plaque array assay.

The plaque aggregates (including cholesterol plaque aggregates, phospholipid plaque aggregates, Abeta plaque aggregates, hybrid plaque aggregates and the like disclosed herein are water soluble. The self-formed plaque particles and the plaque particles disclosed herein are water insoluble. The aggregates of various Abeta peptides disclosed herein as Abeta aggregates generally referred to in the literature as oligomers. As disclosed herein, an array or a panel refer to a plurality of plaque aggregates or self-formed plaque particles.

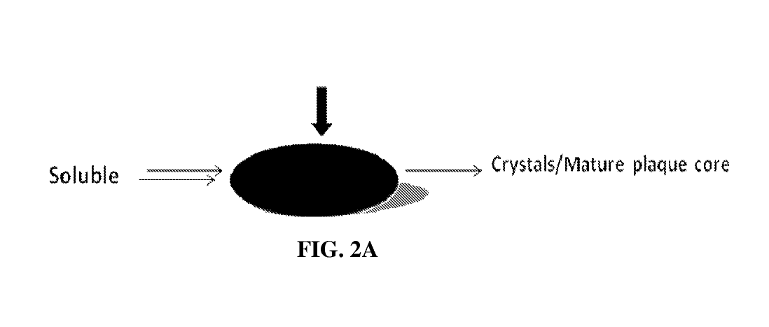

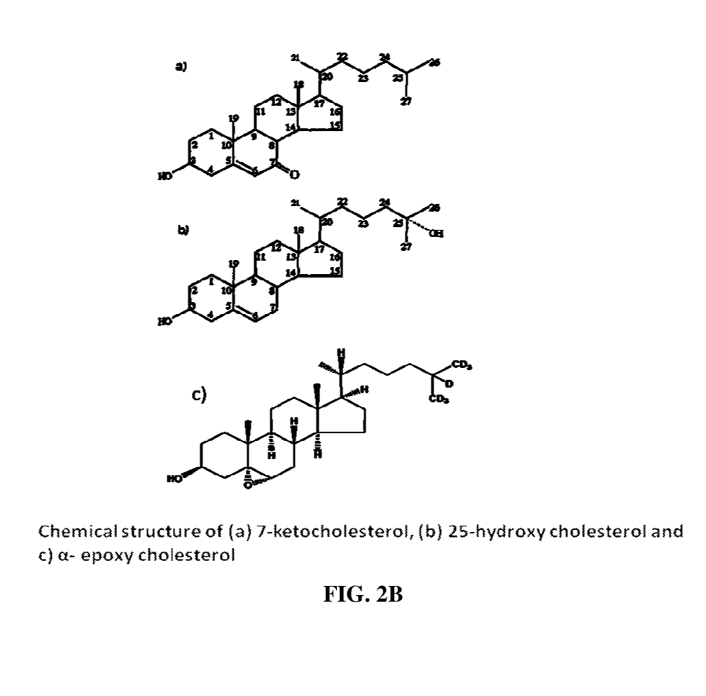

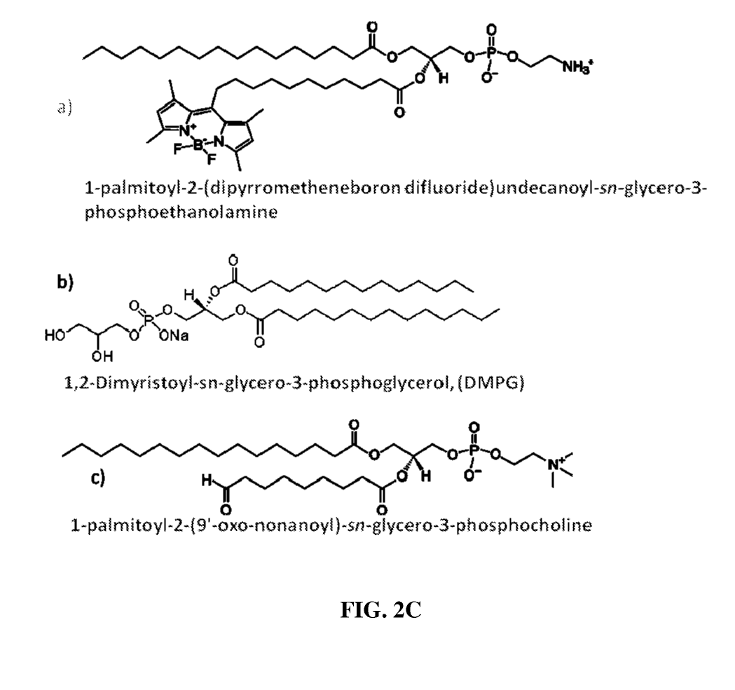

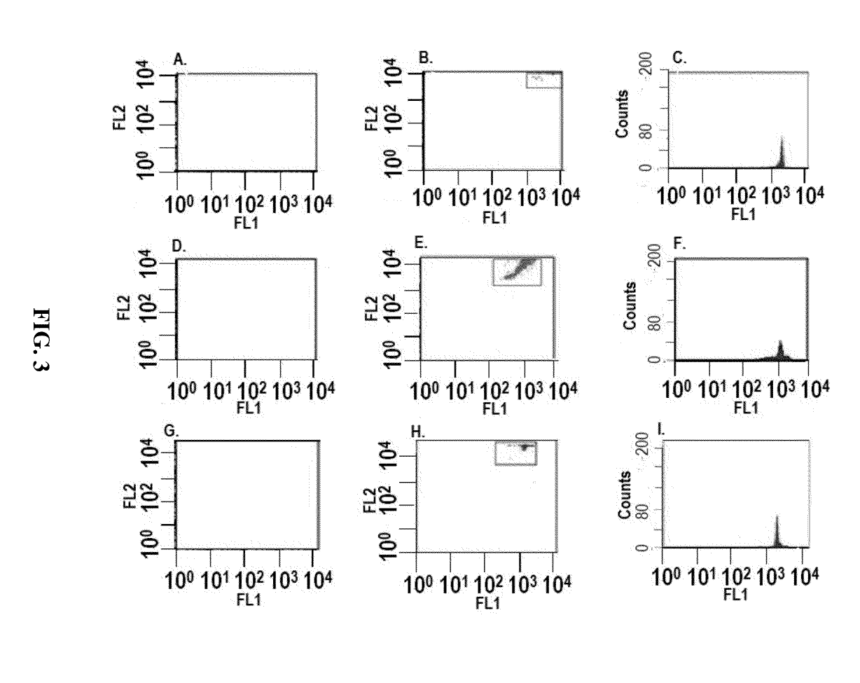

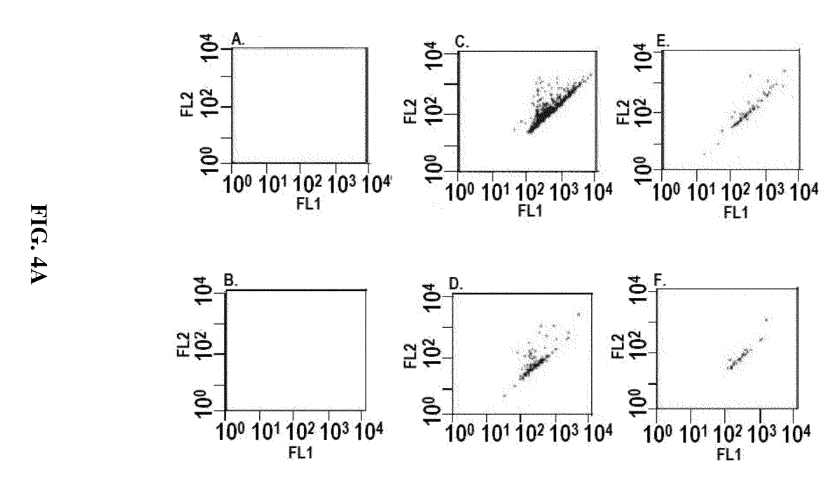

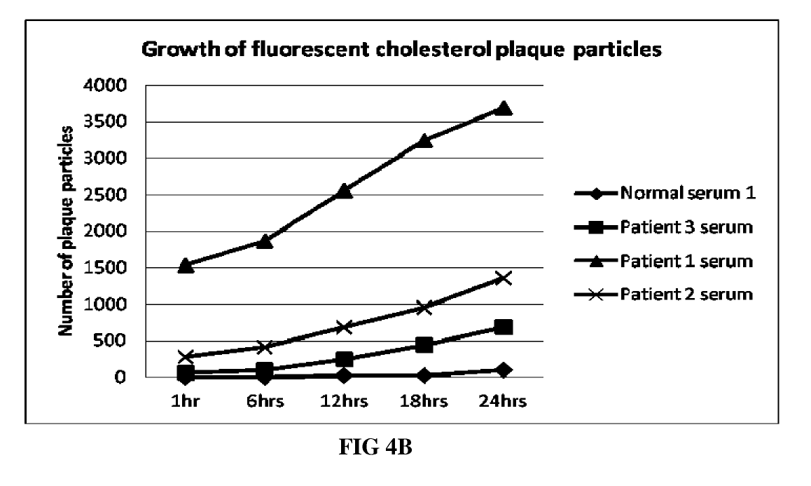

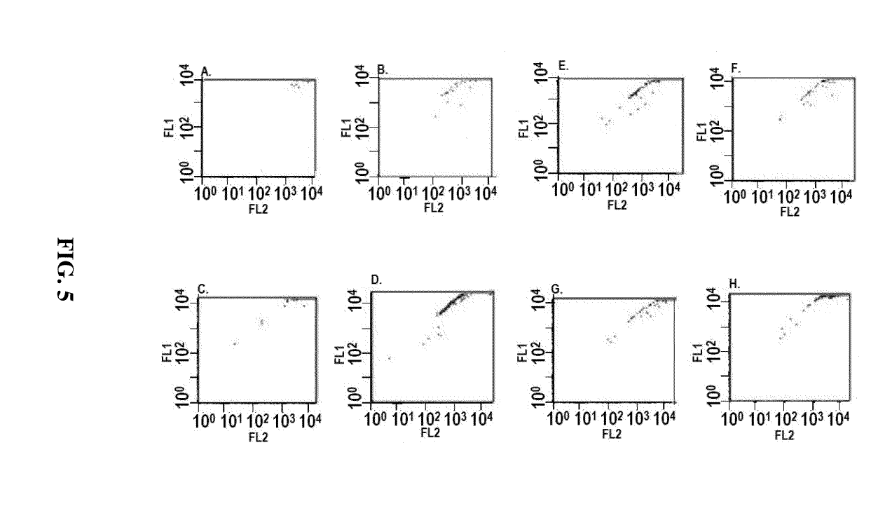

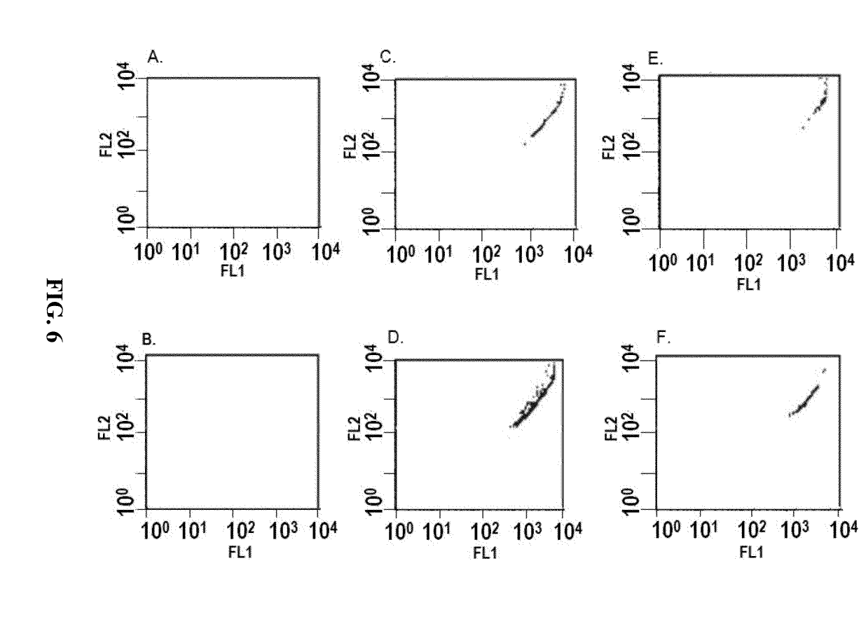

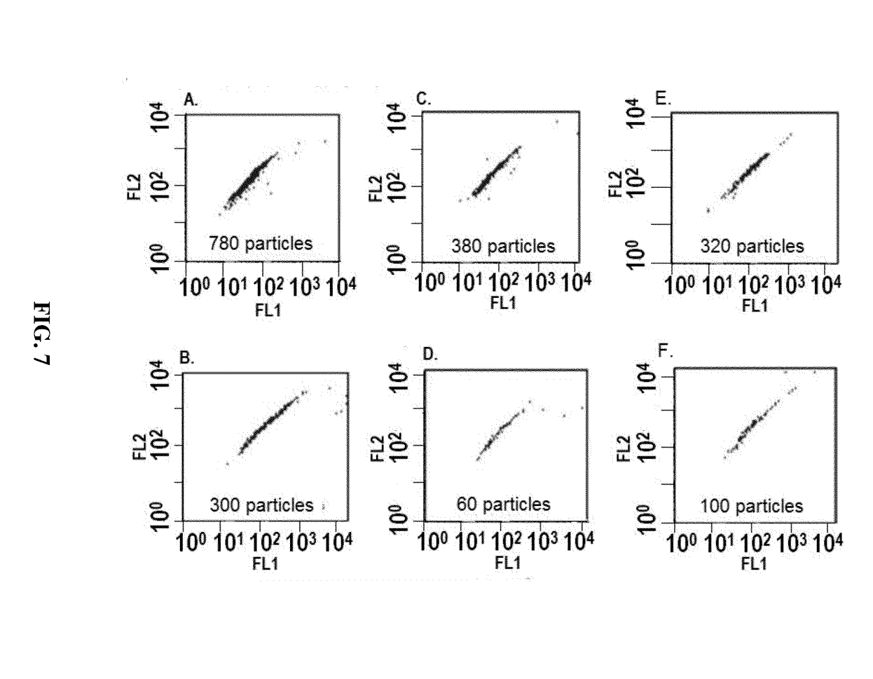

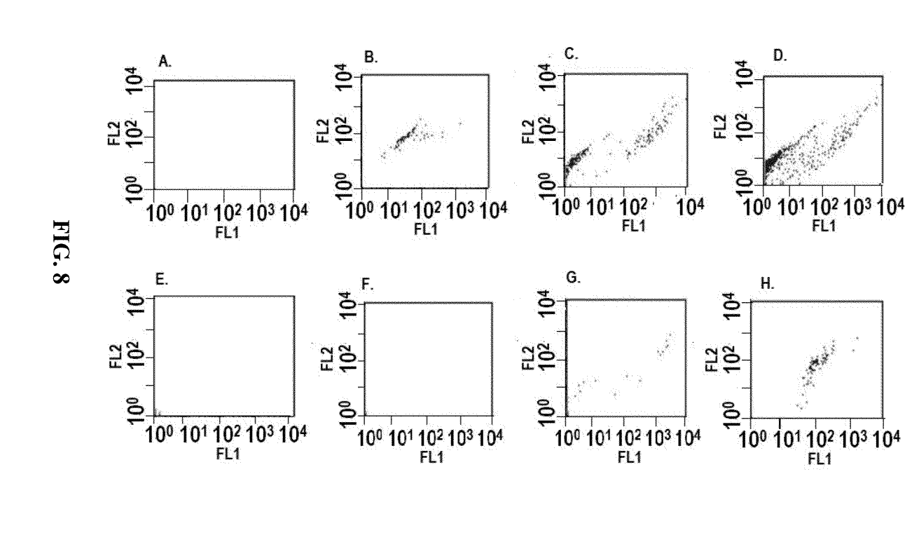

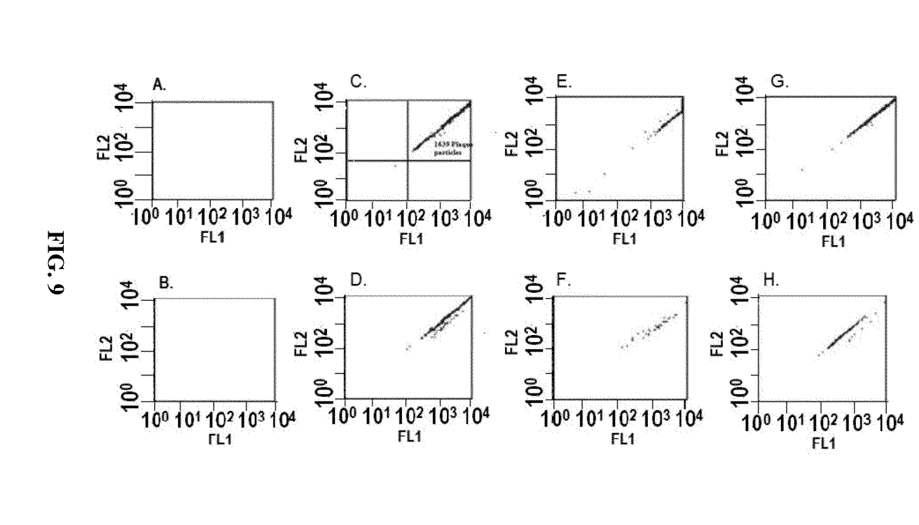

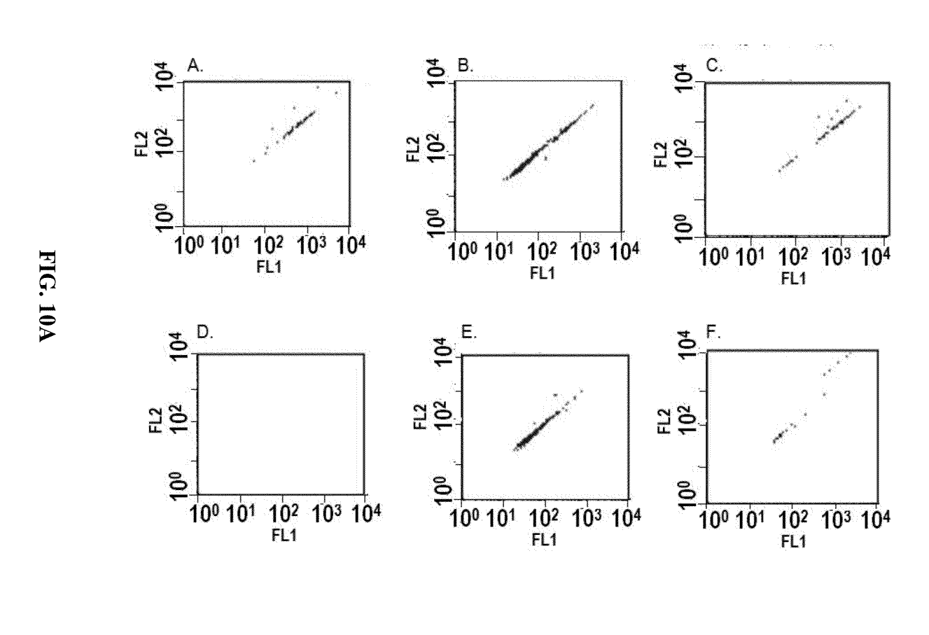

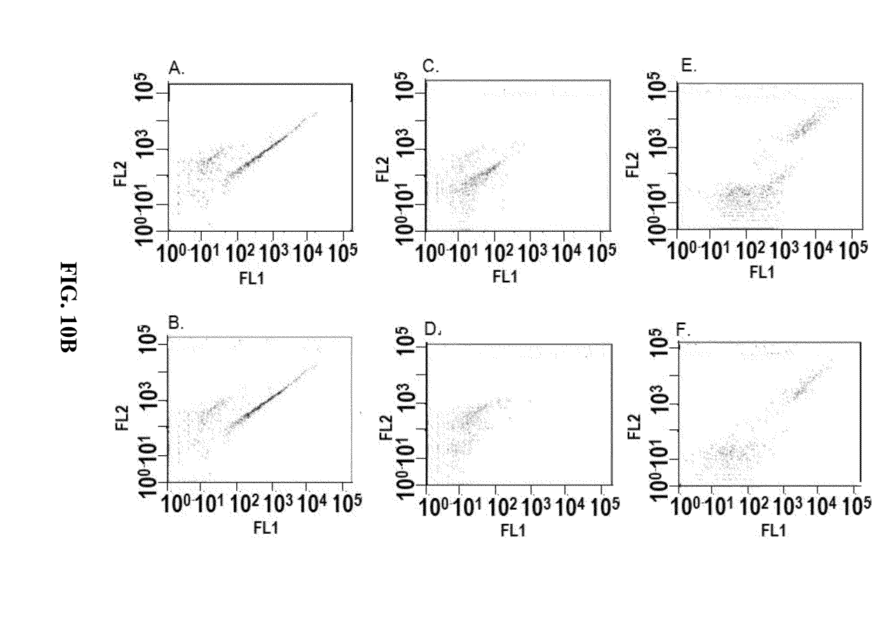

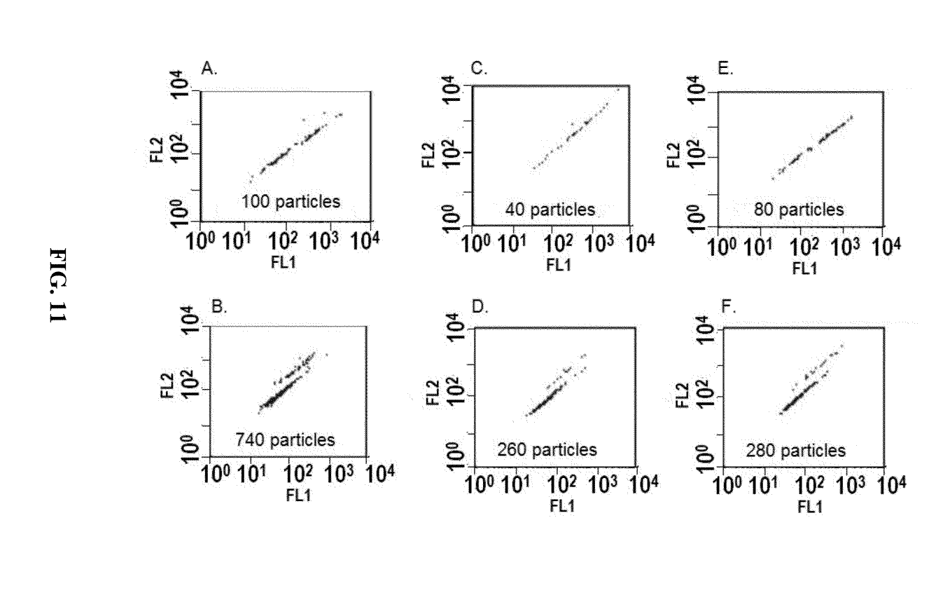

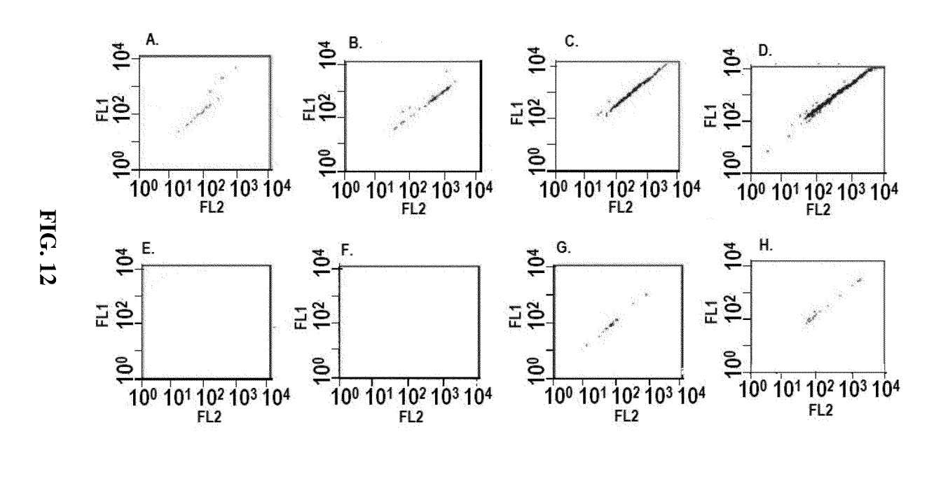

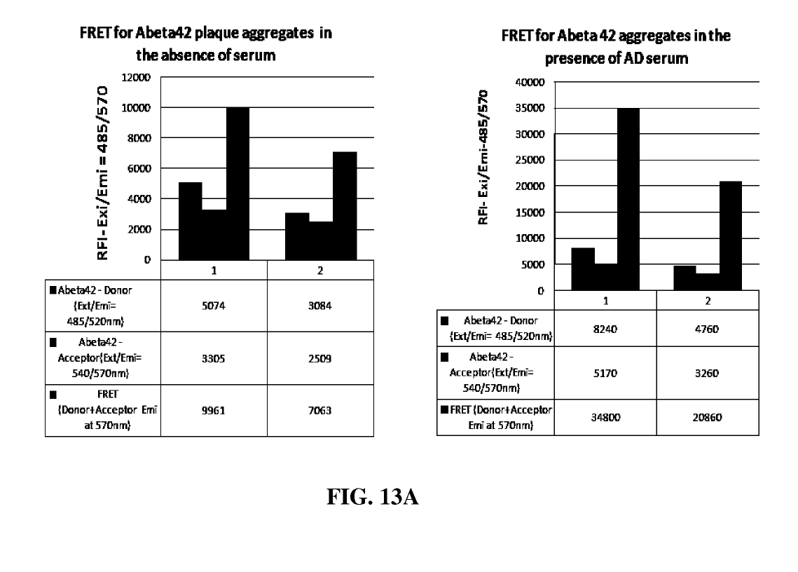

In some embodiments disclosed herein the plaque array method uses building block materials that are normally present in atherosclerotic and amyloid plaques, including lipids, proteins, cholesterol, calcium, amyloid peptides, endothelial cells, bacteria, and minerals. These components exist inside the in vivo plaques as soluble aggregates and mature plaque/crystalline forms and contribute to origin and progression of the plaque-associated diseases (FIG. 2A). Accordingly, the plaque array method uses one or more of these plaque aggregates or self-formed plaque particles for examining in vitro plaque particle formation when contacted with biological sample.

Some embodiments disclosed herein relate to atherosclerosis. In these embodiments, the plaque aggregates or self-formed plaque particles used to screen biofluids or biological samples effect on plaque particle formation may comprise one or more of the following: protein, protein derivative, cholesterol, cholesterol derivatives—(cholestatrienol(=cholesta-5,7,9(11)-triene-3β-ol), 22-NBD-cholesterol(=22-(N-(7-nitrobenz-2-oxa-1,3-diazol-4-yl)amino)-23,24-bisnor-5-cholen-3-ol),α-epoxycholesterol-Cholestan-5α,6α epoxy-3beta diol; 35-hydroxy cholesterol; 7-keto cholesterol, cholesterol monohydrate etc. (Examples shown in FIG. 2B), lipid, or lipid derivatives Lysphosphotidylcholine; C6-NBD-phosphatidylcholine (C6-NBD-PC), C12-NBD-phosphatidylcholine (C12-NBD-PC), DMPG-1,2 dimyristoyl-sn-glycero-3 phosphocholine; 1-palmitoyl-2-(dipyrrometheneboron difluoride)undecanoyl-sn-glycero-3-phosphoethanolamine, 1-palmitoyl-2-(9′-oxo-nonanoyl)-sn-glycero-3-phosphocholine, phosphatidylethanolamine etc, FIG. 2C).

Some embodiments disclosed herein relate to amyloidosis. In these embodiments, the plaque aggregates or self-formed plaque particles used to screen biofluids or biological samples effect on plaque particle formation may comprise one or more of the following: Abeta peptides and derivatives

### Abeta 1-42 DAEFRHDSGYEVHHQKLVFFAEDVGSNKGAIIGLMVGGVVIA;

### Abeta 1-28 DAEFRHDSGYEVHHQKLVFFAEDVGSNK;

### Abeta 1-17 DAEFRHDSGYEVHHQKL;

### Abeta 22-35 EDVGSNKGAIIGLM;

### Amyloid (1-42 S26C) DAEFRHDSGYEVHHQKLVFEAEDVGCNKGAIIGLMVGGVVIA;

### Amyloid (1-42); E22V-DAEFRHDSGYEVHHQKLVFFAVDVGSNKGAIIGLMVGGVVIA;

Amyloid (1-42); N27A-DAEFRHDSGYEVHHQKLVFFAEDVGSAKGAIIGLMVGGVVIA etc), Synuclein, prion, Amylin, Tau, phospholipids, cholesterol crystals, Serum Amyloid A, Beta Microglobulin, lysozyme, insulin, or super dioxide dismutase. In some embodiments, the method comprises a mixture of fluorescently-labeled calcium-phosphate (CP), lipids and cholesterol.

In some embodiments, plaque aggregates or self-formed plaque particles comprise at least one component known to persons of ordinary skill in the art to be present in in vivo formed plaques in subjects with symptomatic and asymptomatic amyloidosis. In these embodiments, the component may be linked to a detectable label. In other embodiments, plaque aggregates or self-formed plaque particles comprise Abeta-42 peptides. In yet other embodiments, plaque aggregates or self-formed plaque particles comprise Abeta-28 peptides.

In some embodiments, plaque aggregates or self-formed plaque particles comprise at least one component known to persons of ordinary skill in the art to be present in in vivo formed plaques in subjects with atherosclerosis. In these embodiments, the component of may be linked to a detectable label. In other embodiments, plaque aggregates or self-formed plaque particles comprise cholesterol or its derivatives. In yet other embodiments, plaque aggregates or self-formed plaque particles comprise phospholipid or its derivatives. In some embodiments, the plaque aggregates comprise a single component while in other embodiments they are hybrid aggregates and comprise more than one component. In some embodiments, the self-formed plaque particles comprise a single component while in other embodiments they are hybrid self-formed plaque particles and comprise more than one component.

In some embodiments, the plaque aggregates and self-formed plaque particles are prepared in phosphate buffered saline (PBS) or phosphate buffers. A person of ordinary skill in the art would recognize that any suitable aqueous solution may be used instead. In some embodiments, the plaque aggregates or self-formed plaque particles are prepared using organic solvents such as alcohol. In some embodiments, the reactions forming plaque aggregates and self-formed plaque particles are performed at 37° C. In other embodiments, the reaction is performed at a temperature and a time which are appropriate for progression of a reaction. In some embodiments the reactions using the plaque aggregates and self-formed plaque particles in diagnostic or drug discovery or development or other context are performed at 37° C. In other embodiments, the reaction is performed at a temperature and a time which are appropriate for progress of a reaction.

The formation of Abeta plaque aggregates is determined by the concentration of the peptides in the buffer, incubation time at 37° C. and presence of metal ions such as copper, iron, aluminium and zinc. We found that incubation of Abeta peptides at 37° C. up to 6 hrs is sufficient to prepare plaque aggregates. Incubation at 37° C. for 24 to 48 hrs produces self-formed Abeta plaque particles. Similarly, cholesterol or phospholipid plaque aggregates are prepared (0 hr) in Phosphate buffered saline (PBS) and used for plaque array assay. Incubation at 37° C. for 12 to 48 hrs leads to the formation of self-formed cholesterol or phospholipid plaque particles. The formation of plaque particles from plaque aggregates in the absence and presence of biofluids is determined by the concentration of the plaque aggregates and incubation time at 37° C. The resulting self-formed plaque particles or plaque particles formed in the serum are insoluble in water, phosphate buffers, Tris-HCL buffers and the like.

In one aspect, the invention is embodied in a method for formation of plaque particles from the plaque aggregates or from self-formed plaque particles. In some embodiments disclosed herein, the formation of plaque particles is accelerated in the presence of biological samples including biofluids. In other embodiments, the formation of plaque particles is accelerated by artificial growth medium, chemical medium and the like.

Any biological sample may be tested according to the disclosed methods. Such a sample may be cells, tissue, blood, urine, semen, or a fraction thereof (e.g., plasma, serum, urine supernatant, urine cell pellet or prostate cells), which may be obtained from a patient or other source of biological material, e.g., autopsy sample or forensic material. Prior to contacting the plaque aggregates or self-formed plaque particles, the sample may be processed to isolate or enrich the sample for the desired molecules using a variety of standard laboratory practices may be used for this purpose, such as, e.g., centrifugation, immunocapture, cell lysis. Biofluid is one category of biological sample. As disclosed herein, the term biofluid is a fluid biological sample and is used interchangeably with the term biological fluid. While the biofluid used in the Examples disclosed herein is serum from human subjects, in some embodiments the biofluid may comprise plasma or saliva. In other embodiments the biofluid may comprise urine, or cerebrospinal fluid. In yet other embodiments the biofluid may comprise blood.

Biological samples may be obtained from animals (including humans) and encompass fluids, solids, tissues, and gases. Biological samples include cerebrospinal fluid, sputum, bronchial washing, bronchial aspirates, urine, lymph fluids, and various external secretions of the respiratory, intestinal and genitourinary tracts, tears, saliva, milk, biological fluids such as cell culture supernatants, tissue, cell, and the like.

In some embodiments, the self-formed plaque particles comprise a linked detectable label which is be detected using fluorescence, luminescence, colorimetry and the like. In another embodiment, the invention comprises an array method for in vitro formation of atherosclerotic or amyloid plaque particles and indirect way of detection of plaque particles or plaque particle sub-types using fluorescent dye or proteins or fluorescently or luminescently-labeled antibodies, the method comprising first converting organic or inorganic molecules into plaque aggregates (0 hr) or self-formed plaque particles (24 hrs), wherein the organic or inorganic molecules is chosen from the group consisting of unlabeled cholesterol or its derivatives or lipid or its derivatives or Abeta-42 or its derivatives, contacting the plaque aggregates or self-formed plaque particles with biological sample so the plaque aggregates interact with the proteins, lipids or carbohydrates and other molecules present in the biological sample leading to in vitro formation of plaque particles. The detection of the resulting plaque particles or plaque particles sub-types employs fluorescently or luminescently-labeled antibodies that bind to components of the plaque particles. This is an indirect method of detecting plaque particles and sub-types that will help to identify the molecules that are attached to the plaque particles and using those molecules as biomarkers for predicting atherosclerosis and AD.

In some embodiments, the in vitro formed plaque particles resemble a plaque associated with, atherosclerosis, Alzheimer's disease, Autism, Parkinson's disease, multiple sclerosis, osteoarthritis, Mad Cow Sponsiform, Type II diabetes, dementia, systemic amyloidosis, dialysis-related amyloidosis, lysozyme amyloidosis, insulin-related amyloidosis, and/or amyotrophic lateral sclerosis.

In certain embodiments, the present invention comprises a method of diagnosing, categorizing, evaluating, quantitating, or predicting atherosclerotic disease or amyloid diseases including Alzheimer's disease in a test subject. Such method may comprise: (a) contacting the blood, plasma, serum, urine, saliva, cerebral spinal fluid of a test subject with a plurality of one or more luminescence or fluorescently-labeled plaque aggregates or self-formed plaque particles; and employing a device to detect the detectable label and identify molecules within the blood, plasma or serum that accelerate plaque particle formation of said one or more plaque aggregates or self-formed plaque particles

The present invention may comprise a method of diagnosing, detecting, analyzing, evaluating or administrating a therapeutically effective dose of a drug, biological compounds, proteins, antibodies or chemical compound to a subject, wherein said drug, biological compound, or chemical compound has been previously screened for its ability to bind, penetrate, disassemble, disrupt, or prevent atherosclerotic plaque or amyloid plaque.

In another embodiment, the invention comprises an array method for identification of molecules present in the biofluids that contribute to assembly of in vitro plaque particles. The cholesterol or phospholipids or amyloid peptide plaque aggregates interact with the proteins, lipids or carbohydrates and other molecules present in the human serum or plasma leading to in vitro formation of plaque particles. Mass spectrometry and proteomics analysis may help to identify the molecules that are attached to the plaque particles and involved in the plaque assembly process. These molecules may be used as biomarkers for detecting the course of in vivo atherosclerosis and amyloid disease development and progression.

In some embodiments disclosed herein the plaque array technology permits the discovery of both novel mechanisms and molecules that catalyze the accelerated plaque particle assembly when treated with the biological samples including biofluids. In some embodiments the plaque array enables the evaluation of the pathogenicity of plaques of varying compositions.

The present invention also embodies a plaque array kit to aid in the diagnosis, prediction, prognosis, or detection of a plaque-associated disease such as AD and atherosclerosis. The method may also comprise obtaining or generating a designation of the risk potential of AD or atherosclerosis against constituent of the panel of plaque particles. In some embodiments, the kit comprises one or more molecules for preparing plaque aggregates, or self-formed plaque particles or plaque particles as described herein. In other embodiments, the kit includes composition of one or more plaque aggregates or self-formed plaque particles or plaque particles with a carrier, e.g. salt, buffer, booster and the like. In other embodiments, the kit further includes reagents of plaque array assay and detection of plaque particles by flow cytometer or luminescence detector.

The present invention also includes plaque array kits that can be used for diagnosis, drug discovery and drug development of plaque-associated diseases. In some embodiments instructions teaching the use of the kit according to the various methods and approaches described herein are provided. Such kits may also include information, such as scientific literature references, package insert materials, clinical trial results, and/or summaries of these and the like, which indicate or establish the activities and/or advantages of the agent. Such information may be based on the results of various studies, for example, biochemical plaque assays, studies using experimental animals involving in vivo models and studies based on human clinical trials. Kits described herein can be provided, marketed and/or promoted to health providers, including physicians, nurses, pharmacists, formulary officials, and the like.

In another embodiment, the invention involves the use of the plaque array method disclosed herein to screen agents for the inhibition or stimulation of the in vitro formation of the plaque particles. As such, agents including but not limited to chemical compounds, small molecule compounds, therapeutic drugs, biological molecules, oligomers, ligands, proteins, antibodies or other components, capable of binding the plaque aggregates or self-formed plaque particles or plaque particles in the presence or absence of biofluids, preventing their assembly, disassembling these aggregates or self-formed plaque particles or plaque particles once already formed, or reducing their pathogenic properties, are tested for their potential as therapeutic leads for diagnosing, preventing, treating, and/or curing amyloid plaque diseases. Since the methods or processes disclosed herein are capable of isolating the steps of in vitro plaque particle formation, anti-plaque agents targeting different stages of plaque development are also capable of being identified. The term “anti-plaque agents” and “anti-plaque therapeutics are used interchangeably herein and refer to compounds or drugs which are effective in a) dissolving, inhibiting or disrupting the architecture, or structure of a plaque aggregates or self-formed plaque particles or plaque particles described herein, and/or b) inhibiting, preventing, or alleviating the detrimental effects that the plaque may have on other cells, tissues or organs of humans.

In certain embodiments, the present invention comprises a method for screening drug, chemical or biological compounds including proteins and antibodies controlling formation of in vitro plaque particles when plaque aggregates or self-formed plaque particles are treated with serum, defined or synthetic medium or plasma samples. This is done by culturing mammalian cells with the at least one plaque aggregates or self-formed plaque particles described herein causing the cells to express morphologic changes, cell death, inflammation, DNA damage, aging or age related degenerative process; and then using the system to screen drug, chemical or biological compounds that prevent or lessen the formation of plaque particle induced pathological symptoms, cell death or morphological changes in the cells. In certain embodiments, the plaque particles resemble an atherosclerotic plaque or amyloid plaque.

In some embodiments, the present invention comprises a method of profiling or categorizing or evaluating efficacy, testing safety of the drug or drug formulations, nanomaterials. In some embodiments, one or more of the plaque aggregates or self-formed plaque particles described herein are used to evaluate drug efficacy, drug safety, pathogenicity in atherosclerotic or amyloid disorders animal models. The animal may be a mouse, a rat, a pig, a horse, a non human primate, a guinea pig, a hamster, a chicken, a frog, a dog, a sheep, a cow, or a human.

### EXAMPLES

### Example 1

### Overview of the Plaque Array Technology

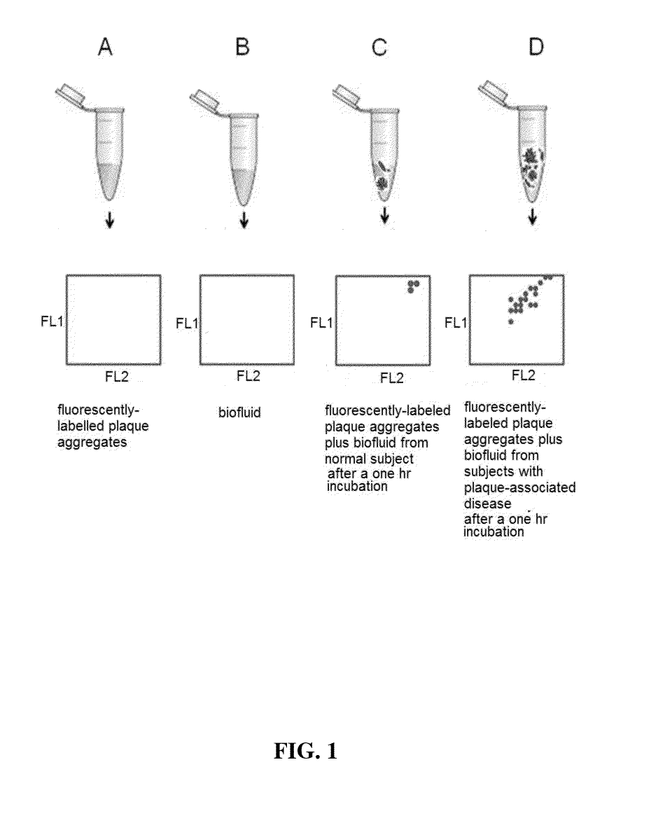

The Example illustrated in FIG. 1 includes both a schematic diagram and steps involved in the development of plaque array method for detection and quantitation of in vitro plaque particle formation catalyzed by molecules present in the biofluids of test subjects. This array method involves three steps: (1) preparation of plaque aggregates or self-formed plaque particles (2) incubation of the plaque aggregates or self-formed plaque particles with biological sample and (3) detection and counting of the resulting plaque particles using a flow cytometer or fluorescence plate reader or luminescence reader or colorimeter or other detection methods

The results of flow cytometry displayed herein are typically presented as one dimensional histogram on a logarithmic scale or two-dimensional displays (dot plot) with logarithmic axes that can extend over a four- to five-decade range. FIG. 1A shows fluorescently-labeled plaque aggregates are not detectable by flow cytometry; FIG. 1B Diluted human biofluids show no detectable plaque particles by flow cytometry; FIG. 1C shows fluorescently-labeled Abeta plaque aggregates or cholesterol plaque aggregates incubated for one hr with biofluids from normal subjects show a small number of plaque particles; FIG. 1D shows fluorescently-labeled Abeta plaque aggregates or cholesterol plaque aggregates incubated for one hr with biofluids from subjects with amyloidoses or atherosclerosis show a significantly greater number of plaque particles in comparison with FIG. 1C. Overall, this shows the serum from subjects with plaque-associated disease accelerates the synthesis of detectable plaque particles from the undetectable plaque aggregate “seeds”.

### Example 2

### Development of a Plaque Array for Plaque-Associated Disease Atherosclerosis

Cholesterol, lipids and calcium crystals are major components present in the atherosclerotic plaques. Specifically, vulnerable atherosclerotic plaques (type-IV, Va) contain significant accumulated cholesterol, lipids and calcified crystals. To develop the plaque array method, first fluorescently-labeled plaque aggregates (0 hr) comprising cholesterol, phospholipids, Abeta peptide and/or calcium-phosphate were prepared. Then, self-formed plaque particles (24 hrs) were synthesized from plaque aggregates by incubating for 24 hrs. The self-formed plaque particles can be detected using a flow cytometer.

The preparation of plaque aggregates from individual molecules was reported earlier (Madasamy, 2009, USPTO Application #: 20090104121). Briefly, 1 mg of fluorescently-labeled cholesterol or cholesterol derivatives (Ex/Em=495 nm/507 nm) was solubilized in 1 mL of 100% alcohol. From this stock solution, 100 μL was taken and mixed in 900 μL of PBS. The transfer of esterified cholesterol molecules from organic medium (alcohol) to PBS buffer caused transformation of individual molecules into cholesterol plaque aggregates (0 hr). The samples were centrifuged for 5 min. at 5000 rpm to remove precipitates, if any, and the supernatant containing soluble aggregates were used for plaque array assay.

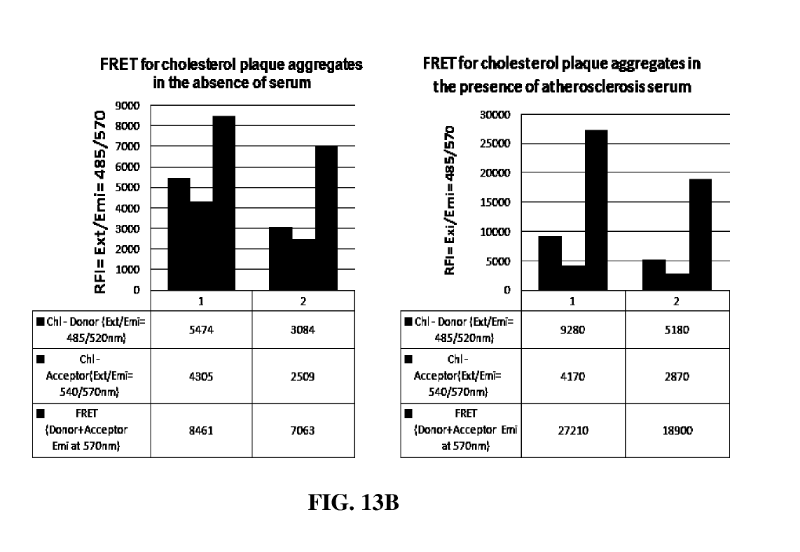

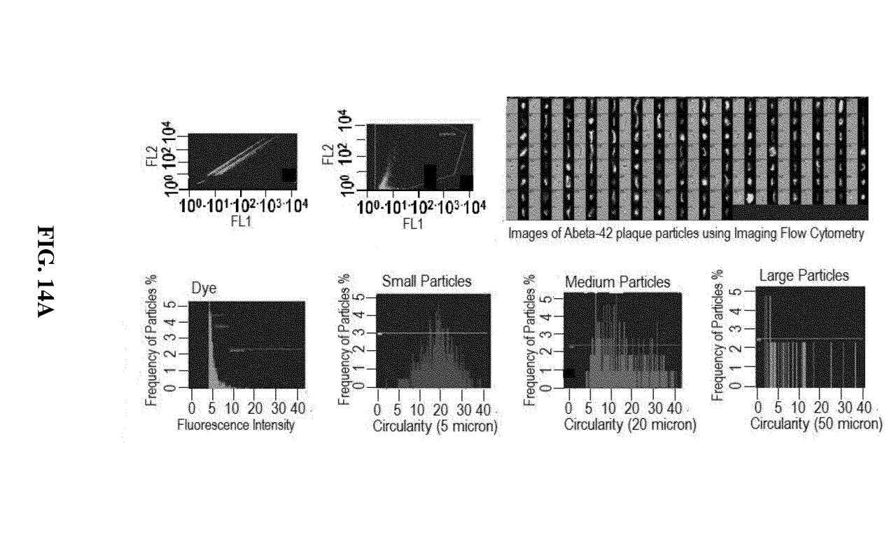

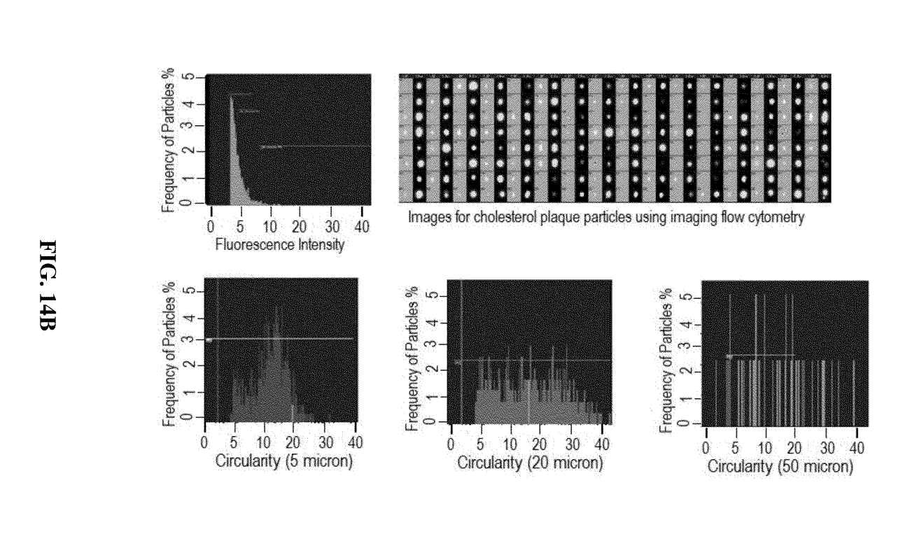

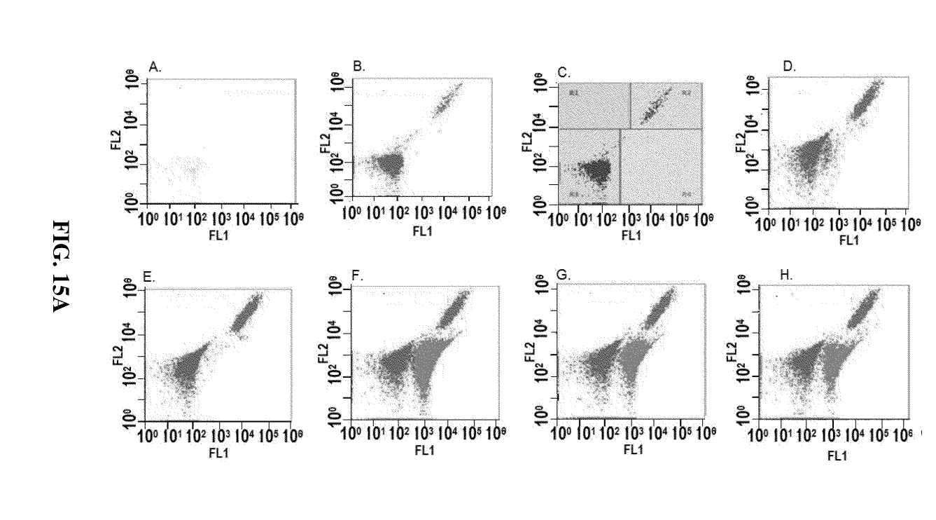

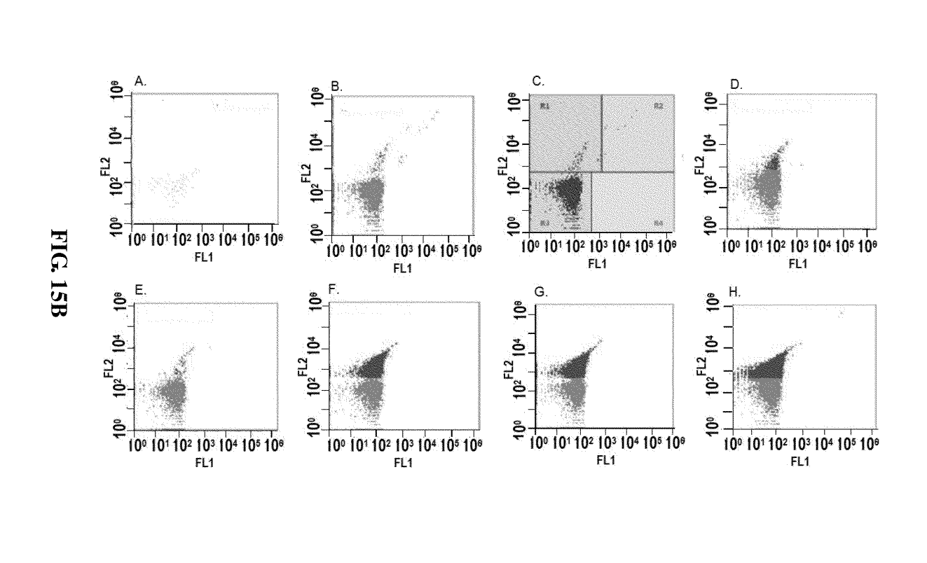

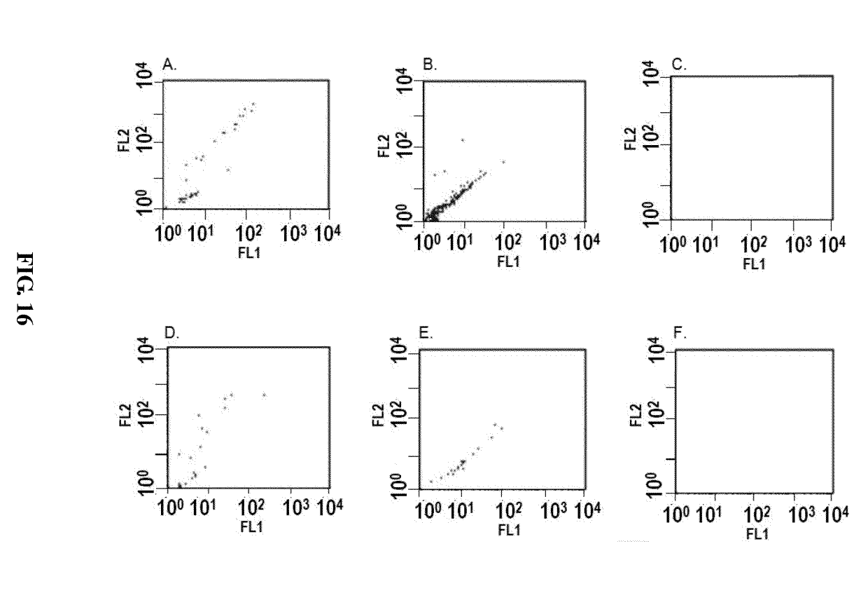

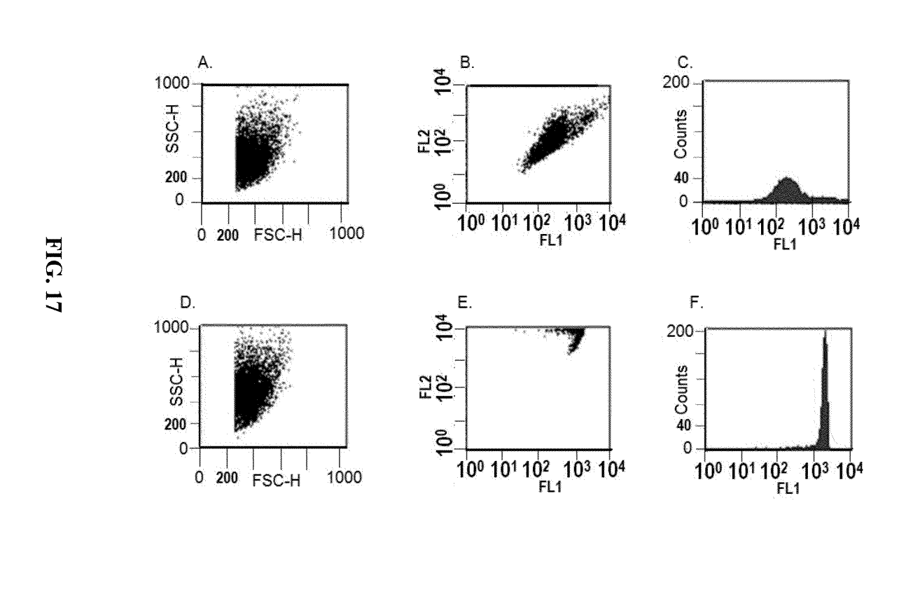

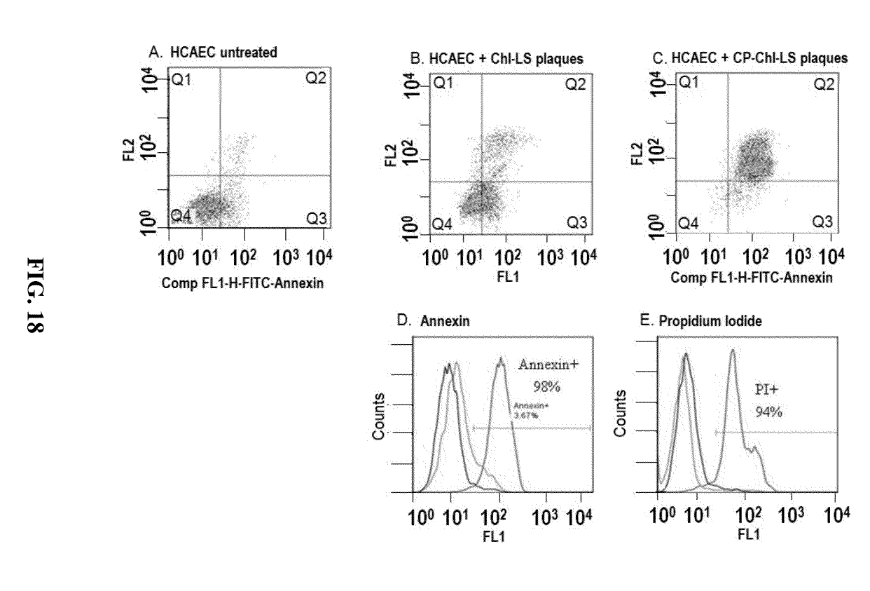

Next, 1-10 μg of the fluorescently-labeled cholesterol plaque aggregate was incubated at 37° C. for 24 hrs to analyze self-formation of plaque particles in the absence of any added biofluids. Both plaque aggregates (0 hr) and self-formed plaque particles (24 hrs) were analyzed using flow cytometer. FIG. 3 (top row) shows a dot plot of fluorescently-labeled cholesterol self-formed plaque particles. Top Row: left display is of fluorescently-labeled cholesterol plaque aggregates (0 hr). Middle and right displays are a two-dimensional dot plot and a one dimensional histogram respectively of self-formed plaque particles synthesized from fluorescently-labeled cholesterol plaque aggregates in a 24 hr incubation period. The particles detected here are designated self-formed plaque particles because their synthesis occurred absent the addition of biofluid from a subject. The results illustrate that cholesterol plaque aggregates were not detected in the flow cytometer whereas plaque aggregates incubated at 37° C. for 24 hrs form detectable self-formed plaque particles. These data suggest that the plaque aggregates (0 hr) are soluble in nature so they were not efficiently detected while passing through the fluorescence detectors in the flow cytometer during sample acquisition process. Conversely, when they are incubated at 37° C. for 24 hrs they aggregate themselves and transform in to self-formed plaque particles in the absence of biofluids that are detected by the flow cytometer.

Next, to prepare fluorescently-labeled phospholipids (LS) plaque aggregates, 1 mg of fluorescently-labeled-phospholipids or its derivatives (Ex/Em=495 nm/507 nm) was solubilized in 1 mL of 100% alcohol. From this stock solution, 100 μL was taken and mixed in 900 μL of PBS. The samples were centrifuged for 5 min. at 5000 rpm to remove precipitate, if any, and the supernatant containing plaque aggregates were used for plaque array assay. The transfer of esterified phospholipids molecules from organic medium (alcohol) to PBS buffer caused transformation of these molecules into phospholipid plaque aggregates. It is notable, when the cholesterol or lipid molecules transform-into soluble or insoluble aggregates they acquire new conformations (Stapronos et al 2003; McCourt et al 1997) thus the resulting plaque aggregates are structurally different from their soluble forms. Next, 1-10 μg of the fluorescently-labeled phospholipid plaque aggregates were incubated at 37° C. for 24 hr to allow synthesis of self-formed plaque particles in the absence of any added biofluids. For detection of both plaque aggregates (0 hr) and self-formed plaque particles (24 hrs) a flow cytometer was used.

FIG. 3 Middle row: left display is of fluorescently-labeled phospholipid plaque aggregates (0 hr). Middle and right displays are a two-dimensional dot plot and a one dimensional histogram, respectively of self-formed plaque particles synthesized from fluorescently-labeled phospholipid plaque aggregates in a 24 hr incubation period. FIG. 3 Bottom row: displays results from experiments where fluorescently-labeled Abeta-42 was used instead of fluorescently-labeled cholesterol and the incubation period was 36 hr instead of the 24 hr used in the cholesterol experiments in this Example.

Together, these results indicate that the 0 hr Ch1, LS and Abeta-42 plaque aggregates are soluble so they were not efficiently detected by flow cytometry. However, when they are incubated at 37° C. for 24 or more hrs they interact and become insoluble plaque particles that can be detected by flow cytometer (FIG. 3).

### Example 3

### Plaque Array Using Fluorescently-Labeled Cholesterol (Ch1) Aggregates to Screen Serum Samples

Different combinations of fluorescently-labeled plaque aggregates or self-formed plaque particles are prepared to mimic various stages of atherosclerosis and used for screening human serum and plasma samples. For each assay using the fluorescently-labeled plaque aggregates, the plasma or serum samples obtained from atherosclerotic subjects and normal healthy subjects are first centrifuged at 5,000 rpm for 5 min. and the supernatants are transferred to new centrifuge tubes. Next, the supernatants are diluted in PBS to make 50% of the serum and plasma samples and used to treat plaque aggregates or self-formed plaque particles to examine if in vitro plaque particle synthesis occurs. Each assay is performed in a 200 μL reaction (100 μL of 50% plasma or serum) and 100 μL (10 μg) of the Ch1-plaque aggregates and the mixtures are incubated at 37° C. for 1 hr. After the incubation, 300 μL sheath fluid is added to the mixture and the samples are used for acquisition (1-2000 events/particles for 1 min) in flow cytometer. FIG. 4A shows the results from an end point assay using flow cytometry analysis of fluorescently-labeled cholesterol plaque aggregates (0 hrs) incubated under various conditions. Plot A displays plaque aggregates after 1 hr incubation (control). Plot B displays plaque aggregates after 1 hr incubation with serum from normal subjects (control). Plots C-F display results of plaque aggregates after 1 hr incubation with serum from four different subjects with atherosclerosis.

A significantly higher number of plaque particles were produced in the incubations with serum samples of the atherosclerosis subjects compared to the controls. We conclude that the presence of serum from subjects with atherosclerosis accelerates the synthesis of plaque particles from plaque aggregates.

A time course study was performed to monitor the formation of plaque particles in the presence and absence of serum from subjects with atherosclerosis. After incubation of fluorescently-labeled cholesterol plaque aggregates (0 hr) with serum, samples were collected at various time points and used for acquisition (1-2000 events/particles for 1 min) in the flow cytometry. FIG. 4B plots the number of plaque particles (y-axis) versus time (x-axis): fluorescently-labeled cholesterol plaque aggregates incubated with serum from normal subjects (diamond); fluorescently-labeled cholesterol plaque aggregates incubated with serum from subjects with atherosclerosis (triangle, cross and squares).

The dot plots reveal a significantly higher number of plaque particles formed in the incubations with serum of subjects with atherosclerosis subjects compared to normal subjects. In addition, the time course shows that the number of plaque particles formed in a sample increased over time from 0 to 24 hrs and analyzed at different time points.

These results suggest that serum samples of the subjects with known history of atherosclerosis related cardiovascular diseases contain factors that ‘catalyze’ in vitro formation of plaque particles from plaque aggregates. In the controls, a small number of plaque particles were detected suggesting the serum samples of the normal subjects may either lack factors that contribute to the plaque particle formation or contain factors that inhibit the in vitro plaque particle formation. It is notable that the human serum or plasma is a complex biological fluid containing approximately 289 proteins and 10⁷ variants of circulating immunoglobulins at a given point in time (Molina H et al, 2005). The accelerated formation of plaque particles may be due to binding of plaque aggregates with a number of molecules including proteins, antibodies, lipids, carbohydrates, metals and metabolites that are present in the serum of atherosclerotic subjects. It would seem likely that substances involved in the formation or assembly of in vitro plaque particles might be specific and correlate with in vivo atherosclerotic plaque development.

### Example 4

### Plaque Array Method Using Fluorescently-Labeled Cholesterol Plaque Particles

Next, fluorescently labeled cholesterol self-formed plaque particles prepared by 24 hrs incubation of cholesterol plaque aggregates (0 hr) at 37° C. were used for incubation with serum samples. For the control experiment, fluorescently-labeled cholesterol plaque particles incubated in PBS in the absence of serum. Each in vitro plaque particle formation assay is performed in a 200 μL reaction (100 μL of 50% plasma or serum and 100 μL (10 μg) of the fluorescently-labeled cholesterol self-formed plaque particles and the mixtures are incubated at 37° C. for 1 hr. After the incubation, 300 μL sheath fluid is added to the mixture and the samples were analyzed in the flow cytometer.

FIG. 5 PAM1, PAM2 etc. are subjects with atherosclerosis. Plot A displays self-formed plaque particles whereas the plots B-H display the results from 1 hr incubation of self-formed plaque particles with serum of subjects with atherosclerosis. Dot plot analysis showed that, unlike plaque aggregates (0 hr), the cholesterol self-formed plaque particles (24 hrs) were detectable in the control plot A. Interestingly, the cholesterol self-formed plaque particles incubated in the serum of subjects with atherosclerosis show significantly greater numbers of plaque particles compared to control. These results are in good agreement with the preceding observations suggesting that serum samples of atherosclerotic subjects contain molecules that accelerate the plaque particle assembly.

### Example 5

### Plaque Array Using Fluorescently-Labeled Phospholipids (LS) Plaque Aggregates

Next, to further examine whether accelerated plaque particle synthesis observed with the Ch1-plaque aggregates is unique mechanism or common to other lipid plaque aggregates, fluorescently-labeled LS-plaque aggregates were prepared and used for screening the diluted human serum and plasma samples. Experiments were performed with serum samples collected from subjects identified for atherosclerosis indications and normal healthy subjects. For control experiment, fluorescently-labeled plaque aggregates were incubated in PBS and not treated with the serum. Each in vitro plaque particle formation assay is performed in a 200 μL reaction (100 μL of 50% plasma or serum and 100 μL (10 μg) of the LS-plaque aggregates and the mixtures are incubated at 37° C. for 1 hr. After the incubation, 300 μL sheath fluid is added to the mixture and the samples were analyzed by flow cytometry.

As shown in FIG. 6, Patients 1, 2, 3 and 4 are subjects with atherosclerosis. Plot A) displays fluorescently-labeled phospholipid plaque aggregates incubated for 1 hr. Plot B) displays fluorescently-labeled phospholipid plaque aggregates incubated for 1 hr with serum from normal subjects. Plots C-F display results of fluorescently-labeled phospholipid plaque aggregates incubated for 1 hr with serum from four different subjects with atherosclerosis.

Dot plot analysis reveals that, as observed earlier with the cholesterol plaque aggregates, phospholipid plaque aggregates were not detectable by flow cytometry (FIG. 6, Plot A) Interestingly, the phospholipid plaque aggregates incubated in the serum of subjects with atherosclerosis show significantly greater numbers of plaque particles (plots C-F) when compared with incubations with serum from normal subjects. As observed earlier with the Ch1 plaque aggregates, these results together strongly suggest that serum samples of atherosclerosis patients contain molecules that contribute to accelerated synthesis of Ch1 and LS-plaque particles from the corresponding plaque aggregates.

### Example 6

### Plaque Array Using IgG-Depleted Serum Against Fluorescently-Labeled Cholesterol Plaque Aggregates

The in vivo atherosclerotic plaques contain immunoglobulin isoforms IgG, IgM and IgA that are co-localized with lipids and cholesterol deposits. However, their role in the manifestations of the atherosclerosis is not completely understood (Hannson G et al, 1984). We have previously identified antibodies in the serum samples of atherosclerotic subjects that bind to self-formed plaque aggregates (Madasamy S, 2009, USPTO Application #: 20090104121). The results described in Examples 3 to 5 demonstrate that serum of subjects with atherosclerosis contain factors that contribute to enhanced in vitro plaque particle formation.

In order to delineate the role of immunoglobulins for in vitro plaque particle formation, IgG-depleted serum of subjects with atherosclerosis was examined using cholesterol plaque aggregates. The IgG depleted serum was prepared by incubating (1 hr at RT) diluted serum in micro-titre plate pre-coated with protein A and protein A/G and pre-blocked with blocking buffer. After incubation, the serum supernatant was screened for its ability to accelerate in vitro plaque particle formation from plaque aggregates. Each assay was performed in a 200 μL reaction (100 μL of diluted serum final concentration is 25% and 100 μL of the aggregates) and the mixtures are incubated at 37° C. for 1 hr for in vitro plaque particle formation. Sheath fluid (300 μL) was added to the mixture and samples were analyzed by flow cytometry for detection and counting of plaque particles.

FIG. 7 shows results from flow cytometric analysis of fluorescently-labeled cholesterol plaque aggregates incubated for 1 hr with: (left) serum from subject with atherosclerosis; (middle) IgG-depleted serum from subject with atherosclerosis that was pretreated with protein A/G and (right) IgG-depleted serum from subject with atherosclerosis that was pretreated with protein A. The IgG-depleted serum samples showed a significant reduction in the number of plaque particle formed when compared with the respective control serum that was not treated with protein A/G for IgG depletion. Analysis of dot plot from IgG-depleted atherosclerosis serum samples showed a reduction of ˜5-50% in plaque particles formation indicating the role of antibodies in plaque particles formation (Table 2).

Abnormal metabolism of cholesterol is implicated in the development of vascular dementia in Alzheimer's disease (Umeda T, et al, 2012). Increasing evidence shows a strong correlation between impaired metabolism of cholesterol and Abeta peptides and that together they play a role in the development of vascular dementia (Pac-Soo C, et al, 2011). Although, the serum samples were collected from subjects with known history of atherosclerosis, we decided to examine whether these subjects had any amyloid plaque related disorders. In order to probe this, as performed earlier, the serum samples were screened using Abeta-42 aggregates and analysis of the resulting dot plot data showed ˜20% of the atherosclerosis subjects were positive for Abeta-42 plaque particles compared to the controls (Table 2). Together, these results strongly suggest the plaque array method could be successfully used to identify and stratify atherosclerosis and AD subjects using different types of plaque aggregates or self-formed plaque particles.

TABLE 2
Summary of artherosclerosis screening of serum samples
for plaque particle formation using plaque array

| Athero- | | | | Protein A/G treated- |
|---|---|---|---|---|
| sclerotic | Cholesterol | Lipid | Abeta-42 | IgG depleted serum. |
| Serum | Plaque | Plaque | Plaque | Cholesterol Plaque |
| samples | particles * | particles * | particles * | particles * |
| Patient 1 | 1340 | 420 | 640 | 760 |
| Patient 2 | 960 | 1600 | 0 | 420 |
| Patient 3 | 300 | 100 | 0 | 60 |
| Patient 4 | 80 | 100 | 0 | 40 |
| Patient 5 | 800 | 560 | 0 | 480 |
| Patient 6 | 1940 | 900 | 100 | 820 |
| Patient 7 | 880 | 800 | 0 | 300 |
| Patient 8 | 280 | 180 | 0 | 220 |
| Patient 9 | 280 | 200 | 0 | 40 |
| Patient 10 | 140 | 80 | 0 | 100 |
| Patient 11 | 860 | 640 | 440 | 280 |
| Patient 12 | 560 | 480 | 0 | 480 |
| Patient 13 | 1860 | 1560 | 0 | 740 |
| Patient 14 | 980 | 680 | 0 | 520 |
| Normal 1 | 40 | 40 | 0 | 20 |
| Normal 2 | 20 | 0 | 20 | 0 |
| Normal 3 | 20 | 0 | 0 | 40 |
| Normal 4 | 0 | 20 | 0 | 20 |

The number of plaque particles shown in the table is for 25 μL serum samples: # of particles × 40 = total number of plaque particles formed/mL. Serum samples collected were from subjects with a previous history of stent fixed, CT scan positive and/or myocardial infarction. These are atherosclerotic subjects or patients. For purposes of atherosclerosis related embodiments disclosed herein normal subjects have no such known history.

### Example 7

### Plaque Array Method Using Serum Samples of Atherosclerotic Mice Model for In Vitro Cholesterol Plaque Particles Formation

Using atherosclerotic mouse models, mice carrying ApoE-gene mutation and normal C57BL/6 mice with same age group were fed with atherogenic diet from 8 weeks to 20 weeks. Serum samples collected at different time points were used for detection of disease progression based on in vitro plaque particle formation from fluorescently-labeled cholesterol plaque aggregates. FIG. 8 shows the results from flow cytometric detection of fluorescently-labeled cholesterol plaque aggregates after 1 hr incubation with serum samples collected at: (left to right) 8 weeks, 12 weeks, 16 weeks and 20 weeks from ApoE-mice (top row) and C57BL/6-mice (bottom row). The results indicate progressive increase in the number of cholesterol plaque particles formed in the incubations of cholesterol plaque aggregates with serum samples of ApoE mutant mice collected over the course of twelve weeks. In the control normal C57BL/6 mice fed with atherogenic diet comparatively small numbers of plaque particle were observed. Together, these results indicate the plaque array method can be successfully used to measure in vivo changes in atherosclerotic mouse model.

Taken together, the plaque array method using fluorescently-labeled Ch1-plaque and LS-plaque aggregates or self-formed plaque particles in combination with serum samples or other biofluids provides a novel serological diagnosis test that helps to measure and profile serum samples from known and unknown subjects. In general, serologic assays are useful both for evaluating the high risk individuals and also complementing risk analysis using data obtained from other diagnosis methods for ultimate decision-making. It is possible the titers of serum molecules that promote plaque particle formation have unique patterns of rise and fall during the longer window period of the atherosclerotic plaque developments. Finally, applying this plaque array method significantly helps rapidly diagnose asymptomatic individuals and take appropriate preventive measures at the early stage of disease development.

### Example 8

### Plaque Array Using Fluorescently-Labeled Abeta-42 Plaque Aggregates

The goal was to examine whether serum from subjects with AD accelerates the synthesis of plaque particles from Abeta-42 aggregates. To prepare fluorescently-labeled Abeta-42 aggregates, 1 mg of fluorescently-labeled Abeta-42 peptide was suspended in 1 mL of PBS and the sample was incubated at 37° C. for 6 hrs. The samples were centrifuged for 5 min. at 5000 rpm to remove precipitate, if any, and the supernatant containing aggregates were used for plaque array assay. Similarly, to prepare unlabeled Abeta-42 aggregates, 1 mg of Abeta-42 peptide was suspended in 1 mL of PBS and the sample was incubated at 37 for 6 hrs. For Abeta-28 aggregates preparation, 1 mg of Abeta-28 was suspended in 1 mL of PBS and the sample was incubated at 37° C. for 6 hrs. The Abeta aggregates prepared by incubation for 6 hrs were not detectable by flow cytometer whereas the self-formed Abeta particles prepared by incubations at 37° C. for 36-48 hrs were detected by flow cytometer. This suggests prolonged incubation of aggregates leads to self-formed plaque particles in the absence of biofluids (FIG. 3, bottom row). Accordingly, the following assays were performed with the Abeta aggregates. Each assay was performed in a 200 μL reaction (100 μL of diluted serum with final concentration of 25% and 100 μL (10 μg) of the aggregates and the mixtures were incubated at 37° C. for 1 hr for in vitro plaque particle formation. For samples containing unlabeled Abeta-42 or Abeta-28 aggregates, after incubation with diluted serum, 10 μL of Thioflavin S (Ex/Em=430 nm/550 nm) fluorescent dye (10 μg) was added and the sample was incubated for an additional 30 min. at 37° C. Following incubation, 300 μL sheath fluid is added to the mixture and the samples are used for acquisition (1-2000 events/particles per min) in flow cytometer.

As shown in FIG. 9, Plot A displays fluorescently-labeled Abeta-42 plaque aggregates incubated for 6 hr (control). Plot B displays fluorescently-labeled Abeta-42 plaque aggregates incubated for 1 hr with serum from normal subjects (control). Plots C-H display results of fluorescently-labeled Abeta-42 plaque aggregates incubated for 1 hr with serum from different subjects with AD. Interestingly, the Abeta-42-plaque aggregates incubated with serum samples of subjects with AD show significantly higher number of plaque particles produced compared to control reactions.

### Example 9

### Plaque Array Using Abeta-42, Abeta-28 and Cholesterol Plaque Aggregates for Screening AD Serum Samples

Next, the serum samples of subjects with AD were screened against three different plaque aggregates to determine the profiles of plaque particle formation among patients. The serum of subjects with AD was tested against Abeta-42 Abeta-28 and cholesterol plaque aggregates for in vitro plaque particle formation according to the protocol described in Example 8 above. As shown in FIG. 10A: Top row: Unlabeled Abeta-42 peptide plaque aggregates were incubated for 1 hr with serum from three subjects with AD. To this mix, Thioflavin S was added and the mixture was incubated for a further 30 min. Flow cytometric analysis of the resulting product shows the serum from subjects with AD accelerates the formation of plaque particles; Bottom row: shows results from corresponding experiment except that unlabeled Abeta-28 plaque aggregates were used instead of unlabeled Abeta 1-42 plaque aggregates. As shown in FIG. 10B: fluorescently-labeled Abeta-42 (left), Abeta-28 peptide (middle) and cholesterol (right) plaque aggregates were incubated for 1 hr with serum from subjects with AD. Flow cytometric analysis of the resulting product shows detectable plaque particles are formed in the Abeta-42 and Abeta-28 plaque aggregates. Analysis of the dot plots result showed significant variations in the number of plaque particles of Abeta-42 versus Abeta-28 produced even with a single patient sample (FIGS. 10A and 10B).

These results suggest each patient serum forms different levels of amyloid and atherosclerotic plaque particle formation which in turn might determine the development and progression of multiple plaque related disorders. The profiles of in vitro plaque particle formation can be applied to distinguish patients affected with Abeta related dementia or vascular dementia caused by abnormal metabolism of Abeta peptide and cholesterol, respectively. These results are in good agreement with our preceding observations with atherosclerotic plaque particle formation and indicate serum samples of subjects with AD contain molecules that catalyze in vitro plaque particle formation.

### Example 10

### Plaque Array Using Antibody Depleted Serum Samples from Subjects with AD

The role of immune system in the development of AD and associated neuroinflammation is well established. Autoantibodies against Abeta peptides have been identified in AD serum samples (Maetzler W, et al, 2011: Hou H, et al, 2012). The results described in the Example 9 demonstrate that serum samples of AD subjects contain factors that contribute to enhanced in vitro plaque particles formation. In order to delineate the role of immunoglobulins in in vitro plaque particle formation, IgG-depleted serum samples of Abeta positive subjects were examined using Abeta-42 aggregates. For antibody response assay, IgG-depleted serum was prepared by incubating diluted serum in microtitre plates pre-coated with protein A and protein A/G and pre-blocked with blocking buffer (1 hr at RT). After incubation, the serum supernatant was incubated with Abeta-42 plaque aggregates and in vitro plaque particle formation was determined. Each assay was performed in a 200 μL reaction and the mixtures were incubated at 37° C. for 1 hr. Sheath fluid (300 μL) was added to the mixture and samples were analyzed by flow cytometry for detection and counting of plaque particles.

Flow cytometric analysis of unlabeled Abeta-42 plaque aggregates incubated for 1 hr with: (left) serum from subject with AD; (middle) IgG-depleted serum i.e. serum from subject with AD that was pretreated with protein A/G and (right) IgG-depleted serum i.e. serum from subject with AD that was pretreated with protein A. The plaque particles were detected using Thioflavin S dye. It was observed that IgG-depleted serum samples showed a significant reduction in the number of plaque particle formation compared with the respective controls that are not treated with protein A/G for IgG-depletion (FIG. 11). Among a number of IgG-depleted AD serum samples tested, different levels of reduction (˜5-50%) in plaque particle formation was observed indicating the role of antibodies in plaque particles formation (Table 3). The data obtained from IgG-depleted serum studies in combination with the data from in vitro Abeta-42, Abeta-28 and cholesterol particles formation provides clinical validation of plaque array method for diagnosing AD and atherosclerosis.

TABLE 3
Summary of AD screening of serum samples for in vitro
plaque particle formation using plaque array.

| Alzheimer's | | | | Protein A/G treated- |
|---|---|---|---|---|
| disease | Abeta-42 | Abeta-28 | Cholesterol | IgG depleted serum. |
| Serum | Plaque | plaque | Plaque | Abeta-42 Plaque |
| samples | particles * | particles * | particles * | particles * |
| Patient 1 | 120 | 0 | 0 | 40 |
| Patient 2 | 800 | 520 | 120 | 280 |
| Patient 3 | 120 | 20 | 0 | 60 |
| Patient 4 | 80 | 0 | 0 | 40 |
| Patient 5 | 580 | 120 | 0 | 320 |
| Patient 6 | 240 | 0 | 0 | 200 |
| Patient 7 | 960 | 0 | 0 | 420 |
| Patient 8 | 420 | 0 | 0 | 220 |
| Patient 9 | 1740 | 860 | 320 | 860 |
| Patient 10 | 580 | 420 | 0 | 420 |
| Patient 11 | 1580 | 680 | 80 | 780 |
| Patient 12 | 850 | 112 | 74 | 400 |
| Patient 13 | 988 | 536 | 477 | 350 |
| Patient 14 | 646 | 436 | 649 | 420 |
| Normal 1 | 20 | 0 | 0 | 20 |
| Normal 2 | 40 | 20 | 0 | 0 |
| Normal 3 | 20 | 0 | 20 | 40 |
| Normal 4 | 20 | 0 | 0 | 0 |

The numbers of plaque particles shown in the table are for 25 μL serum samples: # of particles × 40 = total number of plaque particles formed/mL. Serum samples were from patients with previous history of mild cognitive impairment (1-6) and AD (7-14). These are subjects with AD. Normal subjects in Table 3 and other AD related embodiments disclosed herein do not have these symptoms.

### Example 11

### Plaque Array for Detection of In Vivo Abeta-42 Plaque Formation Using AD Mice Model

AD mice model carrying APPSWE/PS-1 mutant genes and C57BL/6 normal mice with same age group were fed with normal diet from 8 weeks to 20 weeks. Serum samples collected at different time points were used for detection of plaque progression using plaque array method. Abeta-42 plaque aggregates were used to examine in vitro plaque particle formation using serum samples from AD model mice and normal mice. The results indicate progressive increase in the number of Abeta-42 plaque particle formation for AD mice model serum samples that are collected from week 8 to week 20. Compared to the AD mice model, a small number of plaque particle formation was observed for control or normal mice (FIG. 12). As observed earlier with atherosclerotic mice models, the serum samples of other animals can used to detect status of in vivo amyloid plaque development using plaque array method.

### Example 12

### Plaque Array in Combination with FRET for Detection of In Vitro Abeta-42 Plaque Particle Formation

Fluorescence Resonance Energy Transfer (FRET) is a versatile biochemical method widely used to study the interaction between bimolecular (Selvin P R 2000). In order to further examine the self-assembly process of plaque aggregates and their transformation in to self-formed plaque particles in the absence of biofluids, the following experiments were carried out. The aforementioned examples demonstrated that fluorescently-labeled plaque aggregates interact or self-assemble with each other leading to the formation of self-formed plaque particles albeit slowly (FIG. 3). Conversely, when the fluorescently-labeled plaque aggregates are incubated in the serum samples of atherosclerosis or AD patients accelerated in vitro plaque particle formation was observed. Based on this observation, the plaque array in combination with FRET was developed for detection of self-formed plaque particle formation.

For the FRET assay, we used the donor Abeta-42 aggregate labeled with a fluorophore (Ext/Emi=485/520) and the acceptor Abeta-42 plaque aggregates labeled with another fluorophore (Ext/Emi=540/570). When the fluorophore of the donor Abeta-42 aggregates is excited directly at 485 nm, a portion of that energy is acquired by the acceptor Abeta-42 followed by emission at the 570 nm wavelength of the donor fluorophore. Significantly, such energy transfer can only occur when the two dyes are in close spatial proximity (typically less than 100 Å) so the extent of signal will depend on the extent of interaction between the donors and acceptors.

As shown in FIG. 13A, two different concentrations (1=2 μg of plaque aggregates; 2=1 μg of plaque aggregates) of fluorescently-labeled Abeta-42 plaque aggregates incubated for 30 min. at 37 C with PBS (bar graph on left) and with serum of subjects with AD (bar graph on right). Excitation at 485 nm and emissions detected at 520 nm and 570 nm respectively. The left column represents the emission detection of fluorescent from the donor molecule (at 570 nm), the middle column represents fluorescence emission from the acceptor molecule absent the donor (at 570 nm) and the right column represents fluorescence emission from the acceptor molecule in the presence of donor (at 570 nm).

In the control FRET experiment, the Abeta-42 plaque aggregates labeled with two different fluorophores when incubated in PBS samples will self-assemble into plaque particles over time leading to the generation of some signal in an end point FRET assay. In the presence of serum of subjects with AD, the rate of formation Abeta-42 plaque particles from Abeta-42 plaque aggregates is significantly accelerated so an enhanced level signal is detected in an end point FRET assay (FIG. 13A). Accordingly, screening serum samples using plaque array based FRET assay will help to identify AD subjects based on increased FRET signals compared to normal and control assays.

### Example 13

### Plaque Array in Combination with FRET for Detection of In Vitro Plaque Particle Formation

As shown in the FIG. 3, incubation of fluorescently-labeled cholesterol aggregates for 24 hrs in the absence of serum leads to self-formed plaque particles. For the atherosclerotic FRET assay, as described in the Example 12, incubation of two different (donor and acceptor) fluorescently-labeled cholesterol plaque aggregates in the presence of PBS or serum of normal subject lead to reduced interaction between the aggregates thus producing less plaque particles and less FRET signal. Conversely, incubation of two fluorescently-labeled cholesterol aggregates in the presence of serum of atherosclerosis subjects lead to formation of enhanced plaque particles thus producing significantly higher FRET signal compared to controls (FIG. 13B). Accordingly, screening of serum samples using plaque array based FRET assay will help to identify atherosclerosis subjects based on increased FRET signals.

For developing a combination FRET assay to screen both AD and atherosclerosis serum samples using one pair of plaque aggregates, the donor Abeta-42 plaque aggregates was labeled with a fluorophore (Ext/Emi=485/520) and the acceptor cholesterol plaque aggregates was labeled with another fluorophore (Ext/Emi=540/570). In this case, when the fluorophore of the donor Abeta-42 aggregates is excited directly at 485 nm, a portion of that excitation energy is acquired by the acceptor cholesterol followed by emission at the 570 nm wavelength of the donor dye. In the control FRET assay, the Abeta-42 plaque and cholesterol aggregates labeled with fluorophores with two different fluorescent excitation/emissions when incubated in PBS or normal serum samples interact with each other leading to the generation of less plaque particles and less FRET signal. In the presence of AD or atherosclerosis serum samples, the interaction between the abeta-42 plaque aggregates is accelerated to form higher number of plaque particles which in turn lead to generation of enhanced the FRET signal. Accordingly, screening of serum samples using the combination FRET assay will help to identify asymptomatic subjects of atherosclerosis and AD.

### Example 14

### Imaging of In Vitro Abeta-42Plaque Particles for Identification of Sub-Types and Phenotypes Analysis

Next, to further understand the mechanism of plaque particles assembly enhanced by serum samples, the images of the in vitro formed Abeta-42 plaque particles were analyzed. Accordingly, to capture the images of individual Abeta-42 plaque particles, serum samples of the AD subjects and the Abeta-42 plaque aggregates (6 hrs) are incubated at 37° C. for 1 hr. After incubation with diluted serum, 10 μL, of Thioflavin S (Ex/Em=430 nm/550 nm) fluorescent dye (10 μg) was added and the sample was incubated for an additional 30 min. at 37° C. Following incubation, images of Abeta-42 plaque particles were acquired using a Imaging flow cytometer (Amnis Corporation, Seattle, Wash., USA). Analysis of images of plaque particles showed at least are three different sizes of Abeta-42 plaque particles (FIG. 14A). In addition, the image analysis shows that the number of small size (1-3μ) Abeta-42 plaque particle is higher than the numbers observed for medium (5-10μ) and large size (25-50μ) plaque particles.

### Example 15

### Imaging of In Vitro Atherosclerotic Plaque Particles for Identification of Sub-Types and Phenotypes Analysis

To further understand the mechanism of plaque particles assembly in the serum samples the images of the in vitro plaque particles were analyzed. Accordingly, to capture the images of individual cholesterol plaque particles, serum samples of atherosclerotic subjects and fluorescently-labeled cholesterol plaque aggregates are incubated at 37° C. for 1 hr. Acquisition of the resulting samples by Imaging flow cytometer and dot plot analysis showed that there are three major species of cholesterol plaque particles produced as a result of incubation with serum samples (FIG. 14B). The image analysis shows that the numbers of small size (1-3μ) cholesterol plaque particles are higher than the numbers observed for medium (5-10μ) and large size (25-50μ) plaque particles. It is important to note that inside the in vivo atherosclerotic plaques core, the cholesterol particles are, found in three different sizes such as small spherulites (3-5μ), elongated structures (10-30μ) and large irregular deposits (100μ) (Sarig S, et al, 1994). Taken together, these results suggest the composition of in vitro plaque particles and their sub-population could be used as biomarker to determine the course of disease progression in symptomatic and asymptomatic subjects of atherosclerosis and AD.

### Example 16

### Identification of Different Types/Sizes of Cholesterol and Phospholipid Plaque Particles

Next, to further confirm the results (Examples 14 and 15) indicating sub-types of cholesterol and phospholipid plaque particles the following experiments were performed. As aforementioned in Examples 4 to 8, fluorescently-labeled cholesterol and phospholipid plaque aggregates were prepared and used for incubation in the diluted human atherosclerotic serum. In the aforementioned examples (Examples 4 to 8), the samples containing in vitro formed cholesterol and phospholipid plaque particles were acquired using flow cytometer for detection of up to 0 to 2000 events/particles per minute. In the present assay, in order to identify rare/all species of plaque particles, more sample volume was acquired in the flow cytometer to capture greater than 10,000 events/plaque particles per minute.

As shown in FIG. 15A, Top row: The cholesterol plaque particles are shown in the gated dot plots of the R4 region: cholesterol plaque aggregates incubated for 1 hr in the absence of serum (left, first plot), cholesterol plaque aggregates incubated for 48 hr in the absence of serum (left, second dot plot), In the third dot plot from the left, the different populations of particles can be identified in regions R1, R2, R3 and R4. The R4 region was identified as the region of interest in the data acquisition plot and gating allowed collection of these particles and cholesterol plaque aggregates incubated for 1 hr with serum of normal subjects (right); Bottom row left to right: cholesterol plaque aggregates incubated for 1 hr with serum of normal subjects and three plots of cholesterol plaque aggregates incubated for 1 hr with serum of subjects with atherosclerosis. The dot plot analysis of resultant sample acquisition show there are three major species of cholesterol plaque particles formed in the serum samples of the atherosclerosis subjects (FIG. 15A). Among these three species, the plaque particles identified in the gate R4 show up to 500-fold variation in atherosclerotic subject compared to normal subjects. The total number of plaque particles and the sub-types of cholesterol plaque particles detected in the gated dot plots together act as biomarkers that help to predict course of the in vivo atherosclerotic disease origin and progression.

Similarly, in order to identify rare/all species of phospholipid plaque particles, more sample volume was acquired in the flow cytometer to capture greater than 10,000 events/plaque particles per minute. As shown in FIG. 15B, top row: phospholipid plaque aggregates incubated for 1 hr in the absence of serum (left, first plot), phospholipid plaque aggregates incubated for 48 hr in the absence of serum (left, second dot plot), In the third dot plot from the left, the different populations of particles can be identified in regions R1, R2, R3 and R4. The R1 region was identified as the region of interest in the data acquisition plot and gating allowed collection of these particles and cholesterol plaque aggregates incubated for 1 hr with serum of normal subjects (right); Bottom row left to right: phospholipid plaque aggregates incubated for 1 hr with serum of normal subjects and three plots of phospholipid plaque aggregates incubated for 1 hr with serum of subjects with atherosclerosis. The dot plot analysis shows there are two major species of phospholipid plaque particles formed in the serum samples of the atherosclerosis subjects. Among these two major species, the plaque particles identified in the gate R1 of dot plot show up to 500 fold variation in atherosclerotic subject compared to normal subjects. The total number and sub-types of cholesterol and phospholipid plaque particles shown in the gated dot plots together act as biomarkers that help to predict course of the in vivo atherosclerotic disease.

The data collected from multiple plaque array analysis such as detection of accelerated plaque particles formation, counting the plaque particle numbers, role of antibodies in plaque particles formation, the phenotype analysis of plaque particles using imaging and identification of plaque particle sub-types together help to develop “Plaque Fingerprinting” (PF). The application of “Plaque Fingerprints” includes clinical diagnosis, developing companion diagnosis for a particular drug and develops personalized medicine for subjects associated with plaque related disorders including AD and atherosclerosis.

The preceding examples strongly suggest that based on accelerated plaque particles formation both asymptomatic and symptomatic individuals of amyloid diseases including Alzheimer's disease and atherosclerosis disease could be rapidly diagnosed using this non invasive plaque array method. It is anticipated that the profile of accelerated plaque particles formation would vary from normal individual to suspected individuals and comparing their profiles would help to diagnose patients and predict severity of the AD and atherosclerotic plaque development non-invasively. Finally, detecting the asymptomatic individuals using the plaque array method would help to identify the suspected asymptomatic individuals early and treat them with appropriate therapy.

### Example 17

### Screening of Phage Display cDNA, and Peptide Libraries Using Plaque Array Method for Identification of Lead Drug Candidates

The preceding examples clearly proved that serum samples of atherosclerotic and Alzheimer's disease subjects contain molecules that contribute to the accelerated formation of the plaque particles. Accordingly, both the mechanisms of in vitro plaque particles formation in the biofluids and the factors contributing to this process are potential targets for drug discovery. Any drug or drug like molecules that would be identified for disrupting or accelerating or inhibiting these processes would be a novel therapeutic candidate for treatment of atherosclerosis and other amyloid plaques related diseases.

The first objective of the drug discovery efforts is to identify molecules that bind to the plaque aggregates so that the initiation of the plaque particles assembly in the biofluids could be altered. To achieve this goal, screening is carried out using phage display cDNA library of human endothelial cells. For binding of plaque aggregates to phage libraries, fluorescently-labeled cholesterol plaque aggregates (10 μg) or Abeta-42 plaque aggregates (10 μg) are mixed with either cDNA library (pfu 1×10⁶) or phage display peptide library (pfu 1×10⁶). After 30 min. incubation at 37° C., the samples are detected by flow cytometry.

As shown in FIG. 16: Top row: cDNA phage library of human endothelial cells panned with fluorescently-labeled cholesterol plaque aggregates (left); peptide phage display library of human endothelial cells panned with fluorescently-labeled cholesterol plaque aggregates (middle) and fluorescently-labeled cholesterol plaque aggregates (right). Bottom row: cDNA phage library of human endothelial cells panned with Abeta-42 plaque aggregates (left); peptide phage display library of human endothelial cells panned with Abeta-42 plaque aggregates (middle) and Abeta-42 plaque aggregates (right). Phage clones showing positive binding to the plaque aggregates in the dot plot are automatically isolated by sorting and used for further analysis to identify nature of the binding molecules.

A similar approach could be employed to successfully identify small molecules after screening the chemical libraries and positive hits could be characterized and identified by mass spectrometry. The above described results clearly indicate that the plaque array is a powerful drug discovery platform for accelerated drug discovery. Also, the platform enables one to screen molecule libraries to identify novel anti-atherosclerotic and anti-amyloid compounds that would effectively disrupt multiple interactions contributing to in vitro plaque particle formation. Because of its unique and innovative nature, the plaque array based drug discovery platform makes it possible to rapidly identify novel therapeutic modalities to prevent or cure atherosclerosis, Alzheimer's and other plaques related diseases.

### Example 18

### Assembly of Plaque Aggregates or Particles with Human Endothelial Cells

In order to determine the interplay between the plaque components and cells, the fluorescently-labeled plaque aggregates or self-formed plaque particles were incubated with both endothelial cells To achieve this, human coronary artery endothelial cells (HCAECs) are grown to confluence in 75 cm² culture flasks containing 20 mL of endothelial cell growth medium (ECGM). After removing the medium, the cells are scraped off the plate and washed once with ice cold PBS buffer. After centrifugation and cell count, the HCAECs (500,000) were incubated with fluorescently-labeled Ch1 plaque aggregates (5 μg) and LS plaque aggregates (5 μg). After 30 min. incubation at room temperature the plaque aggregate treated cells were detected by flow cytometry. FIG. 17A) dot plot of fluorescently-labeled cholesterol plaque aggregates incubated with HCAECs B) dot plot of fluorescently-labeled cholesterol plaque aggregates incubated with HCAECs C) histogram of fluorescently-labeled cholesterol plaque aggregates incubated with HCAECs D) dot plot of fluorescently-labeled phospholipid plaque aggregates incubated with HCAECs E) dot plot of fluorescently-labeled phospholipid plaque aggregates incubated with HCAECs F) histogram of fluorescently-labeled phospholipid plaque aggregates incubated with HCAECs. Both dot plot and histogram analysis showed that fluorescently-labeled Ch1-plaque aggregates (FIG. 17A, B, C) and LS-plaque aggregates (FIG. 17D, E, F) efficiently bind to HCAECs. These results suggest that the binding of the plaque aggregates directly to the HCAECs may induce several pathological changes such as apoptosis, DNA damage and ageing to the cells.

### Example 19

### HCAECs Binding with Ch1-LS and CP-Ch1-LS to Examine Apoptosis

Next, to probe the consequences of binding of plaque aggregates with the endothelial cells the plaque infected cells were analyzed for apoptosis. First, the hybrid plaque aggregates such as calcium phosphate (CP)-Ch1-LS and Ch1-LS are prepared as reported earlier (Madasamy S, 2009, USPTO Application #: 20090104121) and incubated with the HCAECs. For the control experiment, the calcium containing hybrid plaque aggregates CP-Ch1-LS were incubated with Flu 3 fluorescent dye that specifically binds to calcium moiety of the plaque aggregates. Second, the plaque aggregate treated cells are grown for 12 hrs. After removing the medium, the cells are scraped off the plate and washed once with ice cold PBS buffer. After centrifugation and cell count, the HCAECs (500,000) are assayed for apoptosis using fluorescently-labeled Annexin V (FITC) and propidium iodide (PI) fluorescent DNA binding dye. After 20 min. incubation at room temperature the cells are sorted in the flow cytometer. The dot plot analysis showed that the control cells that were not treated with plaque aggregates showed no significant apoptosis (FIG. 18A), whereas the LS-Ch1 plaque aggregates treated HCAECs showed ˜15% apoptosis (FIG. 18B) and CP-Ch1-LS plaque aggregates treated cells showed significantly higher level of apoptosis (˜94%). This conclusion is supported in the histogram from of the Annexin V assay which shows that CP-Ch1-LS treated cells are 98% apoptosed (18D) and the histogram from the propidium iodide assay shows that CP-Ch1-LS treated cells are 94% apoptosed (18E). Clearly, the binding of CP-Ch1-LS plaque aggregates directly to the HCAECs cause severe pathological symptoms to the cells. This suggests that the in vivo binding of atherosclerotic plaque aggregates to the endothelial cells could lead to their dysfunction and eventually death step in the development of atherosclerotic plaques.

This plaque aggregates infected cell culture system using HCAECs or PBMCs enables assembly of different plaque sub types to mimic atherosclerotic plaques sub types such as pre-atheroma (type 1 to III), containing high lipids content and atheroma type (type IV and Va) containing CP, Ch1, lipids and fibrin clots. Identification of drug compounds that prevent or inhibit the pathogenic effect of plaque aggregates or plaque particles on HCAECs or PBMCS could be successful therapeutic candidates for treating atherosclerosis and other plaques related diseases. In addition, the plaque based HCAEC and PBMC cell culture model system could be used to test efficacy and safety of the anti-atherosclerotic or anti-amyloid drugs.

### OTHER PUBLICATIONS

1. Libby P, Ridker P M, Kansan G K (2011) Progress and challenges in translating the biology of atherosclerosis. Nature, 473, 317-325.
2. Naghavi M, Libby P, Falk E, Casscells S W, Litovsky S et al (2003) From vulnerable plaque to vulnerable patient: A call for new definitions and risk assessment strategies: part I. Circulation. 108, 1664-1672.
3. Greenland et al (2007) Expert Consensus Document on Electron Beam Computed Tomography); Society of Atherosclerosis Imaging and Prevention; Society of Cardiovascular Computed Tomography. Circulation 23; 115(3):402-26.
4. Yankner B A (1996). Mechanisms of neuronal degeneration in Alzheimer's disease. Neuron 16:921-932
5. Selkoe, D J (2003) Folding proteins in fatal ways. Nature 426, 900-904.
6. Dobson, C M (2003) Protein folding and misfolding. Nature 426, 884-890
7. Bell R D and Zlokovic B V (2009) Neurovascular mechanisms and blood-brain barrier disorder in Alzheimer's disease. Acta Neuropathol 118, 103-113
8. Khachaturian, (1985) Diagnosis of Alzheimer's disease. Arch. Neurol. 42: 1097
9. McKhann et al, (1984) Clinical diagnosis of Alzheimer's disease: report of the NINCDS-ADRDA Work Group under the auspices of Department of Health and Human Services Task Force on Alzheimer's Disease. Neurology 34: 939
10. George-Hyslop et al. (990) Genetic linkage studies suggest that Alzheimer's disease is not a single homogeneous disorder. Nature 347: 194.
11. Thies W, Bleiler L; (2012) Alzheimer's Association. Alzheimer's Association report: 2012 Alzheimer's disease facts and figures. Alzheimers Dement. 8:131-168.
12. McKhann G M, Knopman D S, Chertkow H, et al. (2011) The diagnosis of dementia due to Alzheimer's Disease: recommendations from the National Institute on Aging-Alzheimer's Association workgroups on diagnostic guidelines for Alzheimer's Disease. Alzheimers Dement. 7, 263-269.
13. Sperling R A, Aisen P S, Beckett L A, et al (2011) Toward defining the preclinical stages of Alzheimer's Disease: recommendations from the National Institute on Aging-Alzheimer's Association workgroups on diagnostic guidelines for Alzheimer's Disease. Alzheimers Dement. 7(3), 280-292.
14. Stapronos I, Pan X, Rapp J H, Feingold K R (2003) Oxidized cholesterol in the diet is a source of oxidized lipoprotein in the human serum. J. Lipid Res. 44, 705-715
15. McCourt H P, Ashraf K, Dorset D L (1997) X-ray crystal structure of cytotoxic cholesterol: 7-ketocholesterol and 25-hydroxycholesterol. J. Lipid Res. 38, 1014-1021
16. Molina H, Bunkenborg J, Hanumanthu Reddy G., Muthusamy B, Scheel P J, Pandey A. A Proteomic Analysis of Human Hemodialysis Fluid. Mole Cell Proteomics 4.5, 637-650 (2005).
17. Hannson, G et al (1984) Acta Path Microbiol Immunol Scan. Sec A. 92, 429-435.
18. Umeda T, Tomiyama T, Kitajima E, Idomoto T, Nomura S, Lambert M P, Klein W L, Mori H. (2012) Hypercholesterolemia accelerates intraneuronal accumulation of Aβ oligomers resulting in memory impairment in Alzheimer's disease model mice. Life Sci. 2012 Jan. 17. [Epub ahead of print]
19. Pac-Soo C, Lloyd D G, Vizcaychipi M P, Ma D. (2011) Statins: the role in the treatment and prevention of Alzheimer's neurodegeneration. J Alzheimers Dis. 27, 1-10. Yankner B A (1996). Mechanisms of neuronal degeneration in Alzheimer's disease. Neuron 16:921-932
20. Maetzler W, Stapf A K, Schulte C, Hauser A K, Lerche S, Wurster I, Schleicher E, Melms A, Berg D (2011) Serum and cerebrospinal fluid uric acid levels in lewy body disorders: associations with disease occurrence and amyloid-β pathway. J Alzheimers Dis. 26, 171-9.
21. Deng J, Hou H, Giunta B, Mori T, Wang Y J, Fernandez F, Weggen S, Araki W, Obregon D, Tan J. (2012) Autoreactive-Aβ antibodies promote APP β-secretase processing. J Neurochem. 120, 732-40
22. Selvin P R (2000) The renaissance of fluorescence resonance energy transfer. Nat Struct Biol 7: 730-734.
23. Sarig S, Utian W H, Sheean L A, Gorodeski G I (1994a) Distribution of unesterified cholesterol-containing particles in human atherosclerotic lesions. Am. J. Pathol. 146, 139-147.

What is claimed is:

1. A method of detecting plaque particle formation in a subject, the method comprising:
a. preparing at least one plaque aggregate or self-formed plaque particle in vitro wherein the plaque aggregate or self-formed plaque particle is linked to a detectable label;
b. contacting a biological sample from the subject with the at least one plaque aggregate or self-formed plaque particle; and
c. following step b, employing a device to detect the detectable label.

2. The method of claim 1, wherein the biological sample is a biological fluid.

3. The method of claim 2, wherein the biological fluid is selected from the group consisting of: blood, plasma, serum, cerebral spinal fluid, urine and saliva.

4. The method of claim 1 or 2, wherein the contacting of step b results in addition of components to the at least one plaque aggregate or self-formed plaque particle such that at least one plaque particle is formed.

5. The method of claim 4, wherein the at least one plaque particle formed is compared to a plurality of self-formed plaque particles.

6. The method of claim 5, wherein the subject is identified as having, or being at risk of having, a plaque-associated disease if the at least one plaque particle is substantially similar to a self-formed plaque particle among the plurality of self-formed plaque particles.

7. The method of claim 6, wherein the plaque-associated disease is atherosclerosis or amyloidosis.

8. The method of claim 1 wherein the at least one plaque aggregates or a plurality of plaque aggregates is used.

9. The method of claim 1, wherein the label is a fluorescent label or luminescent label or dye.

10. The method of claim 1, wherein the device is a flow cytometer or fluorescence detector or luminescent detector or colorimeter.

11. The method of claim 1, wherein the subject has, is at risk of having, or is suspected of having, atherosclerosis or an amyloidosis including Alzheimer's disease.

12. The method of claim 1, wherein the at least one plaque aggregate or self-formed plaque particle comprises one or more of the following: protein, protein derivative, cholesterol, cholesterol derivative, lipid, lipid derivative, Abeta-42, Abeta derivatives, Synuclein, prion, Amylin, Tau, phospholipids, cholesterol crystals, Serum Amyloid A, Beta Microglobulin, lysozyme, insulin, or super dioxide dismutase, and calcium-phosphate (CP).

13. The method of claim 1, further comprising screening the biological sample with a plurality of plaque aggregates or a pair of plaque aggregates labeled with different fluorophores for generating fluorescence resonance energy transfer (FRET) or a plurality of self-formed plaque particles or a pair of self-formed plaque particles labeled with different fluorophores for generating fluorescence resonance energy transfer (FRET).

14. The method of claim 1, further comprising monitoring the subject by repeating steps a through c at different points over time.

15. The method of claim 4, wherein the plaque particle resemble a plaque associated with atherosclerosis, Alzheimer's disease, Autism, Parkinson's disease, multiple sclerosis, osteoarthritis, Mad Cow Sponsiform, Type II diabetes, dementia, systemic amyloidosis, dialysis-related amyloidosis, lysozyme amyloidosis, insulin-related amyloidosis, and/or amyotrophic lateral sclerosis.

16. A method for detecting plaque particle formation in a subject, comprising:
a. preparing at least one plaque aggregate or self-formed plaque particle;
b. contacting a biological sample from the subject with the at least one plaque aggregate or self-formed plaque particle;
c. contacting the product of step b with detectable label or an antibody-linked detectable label; and
d. following step c, employing a device to detect the detectable label.

17. A method of screening a test agent comprising:
a. preparing at least one plaque aggregate or self-formed plaque particle in vitro wherein the plaque aggregate or self-formed plaque particle is linked to a detectable label;
b. contacting the at least one plaque aggregates or self-formed plaque particles linked to a detectable label with at least one test agents;
c. following step b, employing a device to detect the detectable label.

18. A method of screening a test agent comprising:
a. preparing at least one plaque aggregate or self-formed plaque particle in vitro wherein the plaque aggregate or self-formed plaque particle is linked to a detectable label;
b. culturing mammalian cells with plaque aggregates or self-formed plaque particles linked to a detectable label wherein the mammalian cells express morphologic changes, pathological symptoms, cell adhesion molecules, cytokines and or apoptosis, inflammation;
c. contacting the mammalian cells with at least one test agents; and
d. identifying test agents that prevent or lessen the formation of pathological symptoms or morphological changes in the cells.

19. The method of claim 1, wherein targeting the mechanism of accelerating plaque particles synthesis or plaque particles assembly in treating with biological fluids of human and other animals for drug discovery.

20. A method of biomarker identification in a subject, the method comprising:
a. preparing at least one plaque aggregate or self-formed plaque particle in vitro wherein the plaque aggregate or self-formed plaque particle is linked to a detectable label;
b. contacting a biological sample from the subject with the at least one plaque aggregate or self-formed plaque particle; and
c. following step b, identification of protein or antibody or metabolite or substance from the biological sample that contributed to accelerated plaque particle formation using proteomics or mass spectrometry analysis or the like.

21. The method of claim 17 wherein the at least one test agent is a small molecule or protein or antibody library of test agents.

22. The method of claim 17 wherein the effect of the test agent is to accelerate the formation of plaque particles.

23. The method of claim 17 wherein the effect of the at least one test agent is to reduce or slow or disrupt plaque particle formation.

24. The method of claim 17 using a plurality of test agents further comprising identifying test agents that prevent or disrupt or reduce plaque particle formation.

25. The method of claim 17, further comprising testing the efficacy of the test agent or agents at disrupting plaque particles or reducing the formation of plaque particles or further comprising testing the safety of the test agent.

26. The method of claim 17, wherein the test agent is a nanoparticle or is formulated with a nanoparticle.

27. The method of claim 4, further comprising diagnosing or stratifying subjects based on plaque particle formation, plaque particle sub-types, plaque particle images, plaque particle count, or plaque particle profile.

28. The method of claim 25, further comprising monitoring the efficacy of the test agent in subjects.

29. A method comprising screening blood or blood products for plaque particle formation using a method of any one of claims 1-28.

30. The method of claim 29, wherein the blood or blood products is administered to a recipient subject following the screening or testing wherein the negative result is a finding of few or no new plaques following the contacting of said blood or blood product with the plaque aggregates or self-formed plaque particles.


SEQUENCE LISTING (7 sequences)

SEQ ID NO: 1 - protein, 42 aa, Homo sapiens [sequence omitted]

SEQ ID NO: 2 - protein, 28 aa, Homo sapiens
DAEFRHDSGYEVHHQKLVFFAEDVGSNK

SEQ ID NO: 3 - protein, 17 aa, Homo sapiens
DAEFRHDSGYEVHHQKL

SEQ ID NO: 4 - protein, 14 aa, Homo sapiens
EDVGSNKGAIIGLM

SEQ ID NO: 5 - protein, 42 aa, Artificial Sequence
  SEQ. ID No. 1 mutant S26C
DAEFRHDSGYEVHHQKLVFFAEDVGCNKGAIIGLMVGGVVIA

SEQ ID NO: 6 - protein, 42 aa, Artificial Sequence
  SEQ. ID No. 1 mutant N27A
DAEFRHDSGYEVHHQKLVFFAEDVGSAKGAIIGLMVGGVVIA

SEQ ID NO: 7 - protein, 42 aa, Artificial Sequence
  SEQ. ID No. 1 mutant E22V
DAEFRHDSGYEVHHQKLVFFAVDVGSNKGAIIGLMVGGVVIA